(19) United States
(12) Patent Application Publication
(10) Pub. No.: US 2010/0015709 A1
(43) Pub. Date: Jan. 21, 2010

(54) Regulating Stem Cell Differentiation By Controlling 2D and 3D Matrix Elasticity

(75) Inventors: Florian Rehfeldt, Gleichen (DE); Shenshen Cai, Philadelphia, PA (US); Dennis E. Discher, Philadelphia, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Trustees of the University of Pennsylvania

(21) Appl. No.: 12/355,161

(22) Filed: Jan. 16, 2009

Related U.S. Application Data
(63) Continuation-in-part of application No. 11/351,420, filed on Feb. 10, 2006.
(60) Provisional application No. 61/011,541, filed on Jan. 17, 2008.

(51) Int. Cl. C12N 5/02; C12N 5/00

(52) U.S. Cl. 435/377

(57) ABSTRACT

Provided are methods for the selection and regulation of the mechanical properties of 2D or 3D biocompatible substrates or tissue microenvironments as a technique to regulate in vitro differentiation, cell shape and/or lineage commitment of anchorage-dependent cells, such as mesenchymal stem cells into, e.g., neurogenic-, myogenic-, and osteogenic-type cells. Substrate mechanical properties include elasticity, tension, adhesion, and myosin-based contractile mechanisms. Inhibitors can be introduced to further regulate differentiation.

### CROSS REFERENCE TO RELATED APPLICATIONS

This case is filed as a Continuation-in-Part Application of U.S. patent application Ser. No. 11/351,420, filed Feb. 10, 2006, and this application claims priority to U.S. Provisional Appl. 60/850,931, filed on Oct. 11, 2006, and U.S. Provisional Application 61/011,541, filed Jan. 17, 2008, each of which is incorporated entirely by reference.

### GOVERNMENT SUPPORT

This invention was supported in part by funds obtained from the National Institutes of Health grant numbers 11R21EB004489-01 and 5R01 AR047292-05. The U.S. government may have certain rights in the invention.

### FIELD OF THE DISCLOSURE

The present disclosure relates to differentiating cells based on mechanical properties of the environment of the cell, in particular, elasticity of the surrounding matrix.

### BACKGROUND

Normal tissue cells are generally not viable when suspended in a fluid. Thus, they are “anchorage-dependent” because to grow, such cells must adhere to a solid matrix, varying in stiffness from rigid glass to soft agar, topography, and thickness (e.g., basement membrane). Anchorage-dependent cells, therefore, are no longer viable if dissociated from the solid matrix and suspended in the culture media, even if soluble proteins are added to engage cell adhesion molecules, e.g., integrin-binding RGD peptide.

Fluids are clearly mechanically distinct from solids, which flow when stressed, whereas solids have the ability to resist sustained deformation. In most soft tissues, e.g., skin, muscle, brain, etc., adherent cells together with an extracellular matrix constitute a relatively elastic microenvironment. Macroscopically, elasticity (measured as ‘Pascal’ or newtons/square meters) is evident in the ability of a solid tissue to recover its shape within seconds after mild poking and pinching, or even after sustained compression. At the cellular scale, normal tissue cells probe elasticity as they adhere and pull on their surroundings. Such processes are dependent in part on myosin-based contractility and transcellular adhesions, centered on integrins, cadherins, and perhaps other adhesion molecules, to transmit forces to substrates. Consequently, adhesion complexes and the actomyosin cytoskeleton, whose contractile forces are transmitted through transcellular structures, play key roles in molecular pathways.

Microenvironments and niches appear important in stem cell lineage specification and differentiation as cells can ‘feel’ tissue softness via contractile forces, generated by cross-bridging interactions of actin and myosin filaments. These forces (referred to as traction forces) are transmitted to the substrate, causing wrinkles or strains in thin films or soft gels (Harris et al., Science 208:177 (1980); Oliver et al., J. Cell Biol. 145:589 (1999); Marganski et al., Methods Enzymol. 361:197 (2003); Balaban et al., Nat. Cell Biol. 3:466 (2001); Tan et al., Proc. Natl. Acad. Sci. USA 100:1484 (2003)). The cell, in turn, responds to the resistance of the substrate by adjusting its adhesions, cytoskeleton, and overall state, e.g.—differentiation. Although considerable attention has been directed at the responsiveness of individual differentiated cells to external forces (outside-in) such as stretching and local twisting (Alenghat et al., Sci. STKE 119:pe6 (2002)), there is little understanding of how cell-exerted forces in response to the surrounding microenvironment contribute to signaling pathways effecting contractile mechanisms and ultimately cell state.

For example, adult stem cells, as part of normal regenerative processes, are believed to migrate or circulate and engraft to sites of injury, and will differentiate within these various in vivo microenvironments, ranging from compliant tissue substrates, such as brain or muscle, to rigid tissue substrates, such as bone. Mesenchymal stem cells (MSCs) are pluripotent, anchorage-dependent, and bone marrow-derived cells differentiating into various types of anchorage-dependent cells, including neurons, myoblasts, osteoblasts, and more (Gang et al., Stem Cells 22:617-624 (2004); Gilbert et al., J. Biol. Chem. 277, 2695-2701 (2002); McBeath et al., Developmental Cell 6: 483-495 (2004); Pittenger et al., Science 284:143-147 (1999); Salim et al., J. Biol. Chem. 279:40007-40016 (2004); Tanaka et al., J. Cell Biochem. 93, 454-462 (2004)) via different signaling paths. Soluble factors and cell density clearly influence these differentiation pathways chemically, but variations can also be physical (Gregory et al., Science STKE PE37 (2005); Salasznyk et al., J. Biomed. Biotechnol. 24-34 (2004)). For instance, stem cells adhere and differentiate in soft brain tissue or near rigid bone, and in vitro on soft gels or hard plastic culture dishes. However, compounding MSC-based therapies which consider physical matrix effects are normal wound healing responses, where the formation of fibrotic scar tissue will stiffen the microenvironment, and genetic disorders, such as muscular dystrophy, which increase fibrosis in affected tissues (Engler et al., J. Cell Biol. 166: 877-887 (2004c).

This wide range in substrate stiffness, exacerbated by disease, has been observed in vivo in many differentiated cell types to strongly influence focal adhesions and cytoskeleton (Beningo et al., J. Cell Biol. 153:881-888 (2001); Bershadshy et al., Annu. Rev. Cell Dev. Biol. 19:677-695 (2003); Discher et al., Science, 310:1139-1143 (Nov. 18, 2005); Engler et al., Biophys. J. 86:617-628 (2004a); Engler et al., 2004c, supra; Georges et al., J. Appl. Physiol. 98:1547-1553 (2005); Pelham et al., Proc. Natl. Acad. Sci. USA 94:13661-13665 (1997); Yeung et al., Cell Motil. Cytoskeleton 60:24-34 (2005)) and to be modulated by Ras superfamily proteins and their effectors (Gregory et al., 2005, supra; Paszek et al., Cancer Cell 8:241-254 (2005); Peyton et al., J. Cell Physiol. 204:198-209 (2005)). Rho subfamily members especially are broadly known to regulate the cytoskeleton, cell growth, and transcription, and recent studies of stem cell differentiation are also beginning to implicate cytoskeletal reorganization in vitro (Rodrigues et al., J. Cell Biochem. 93:721-731 (2004)) and Ras superfamily signaling in vivo (Benitah et al., Science 309:933-935 (2005)).

In addition to cell differentiation, the mechanical resistance or elasticity of a tissue cell's surrounding microenvironment adjusts spread morphology and contractile forces (Cukierman et al., Science 294:1708-1712 (2001); Engler et al., 2004a, supra; Flanagan et al., Neuroreport 13:2411-2415 (2002); Tolic-Norrelykke et al., Am. J. Physiol. Cell Physiol. 283:C1254-1266 (2002)), as well as motility and viability (Engler et al., 2004c, supra; Lo et al., Biophys. J 79:144-152 (2000); Peyton et al., 2005, supra; Wang et al., Am. J. Physiol. Cell Physiol. 279:C1345-1350 (2000); Wong et al., Langmuir 19:1908-1913 (2003)), and protein expression and signaling (Beningo et al., 2001, supra; Pelham et al., 1997, supra). The involvement of contractile-effector proteins in sensing implies that cell crawling, and thus MSC's ability migrate or circulate and engraft to sites of injury is also likely to be sensitive to substrate stiffness, as demonstrated in studies of the “cell on gel” effect with epithelial cells (Pelham et al., 1997, supra), fibroblasts (Lo et al., 2000, supra), and smooth muscle cells (Peyton et al., 2005, supra; Zaari et al., Adv. Mater. 16:2133 (2004)). With the latter cell type, crawling speed appears maximal at an intermediate stiffness and is reminiscent of crawling speed versus adhesive ligand concentration (Goodman et al., J. Cell Biol. 109:799 (1989)), mathematically modeled as a shift in the balance between ligand-mediated traction and ligand-mediated anchorage (Zaman et al., Biophys. J. 89:1389 (2005)). Additionally, smooth muscle cells on gels are slowed by inhibition of Rho kinase, suggesting that RhoA activity contributes to the tensions needed to detach any established adhesions at the rear of a motile cell (a process not needed in engulfment) (Jay et al., J. Cell Sci. 108:387 (1995)). The dependence of cell crawling speed and direction on substrate stiffness, particularly gradients in stiffness, is referred to as “durotaxis” (Lo et al., 2000, supra).

Nevertheless, while cells have been shown to respond to externally applied forces (see, e.g., Riveline et al., J. Cell Biol. 153:1175-1186 (2001)), until the present invention there was no suggestion of a relationship between pluripotent cell differentiation and matrix elasticity and how various disease states can complicate the physical remodeling required to decrease elasticity to proper, tissue-relevant levels prior to the use of stem-cell based therapies. Thus prior to the present invention, a need remained in the art to provide a method for regulating the differentiation of mesenchymal stem cells (“MSCs”) into anchorage-dependent cell types, and more specifically methods that are effective on tunable biocompatible substrates. Moreover, similar sensitivity, growth and remodeling principles seem to apply to most anchored cell types, and by regulating differentiation via contractile mechanisms, light may be shed on other matrix-altering pathologies.

### SUMMARY OF THE INVENTION

A normal tissue cell not only applies forces, but also, as demonstrated in the following disclosure, responds through cytoskeleton organization (and other cellular processes) to the resistance that the cell senses, regardless of whether the resistance derives from a normal tissue matrix, synthetic substrate, or even an adjacent cell. Thus, the present invention meets the foregoing identified needs and other purposes by providing methods for regulating differentiation and cell shape of an anchorage-dependent cell.

It is, therefore, an object of the present invention to provide a method for regulating differentiation and cell shape of an anchorage-dependent cell, comprising: selecting, designing, or engineering a substrate or tissue microenvironment having an elasticity defined by elastic constant E; introducing the anchorage-dependent cell onto a substrate or into a microenvironment; and developing the anchorage-dependent cell into a differentiated cell type, wherein shape and lineage commitment (in terms of gene or protein expression, or both) are regulated by the elasticity of the underlying substrate. Depending on the controlled elasticity of the substrate, there is implemented a differentiation of the anchorage-dependent cells into at least one neurogenic, myogenic or osteogenic-type cell.

It is an additional object of the invention to provide a method wherein the subject anchorage-dependent cell is exposed to an inhibiting agent to inhibit expression of a lineage-specific regulator.

It is a further object of the invention to provide a method for regulating differentiation and cell shape of an anchorage-dependent cell, comprising: selecting, designing, or engineering a substrate or tissue microenvironment having an elasticity defined by elastic constant E; and introducing the anchorage-dependent cell onto a substrate or into a microenvironment, and balancing chemo-mechanical energetics localized to cell adhesions against contractile energetics, σ, of the cell that balances cell traction stresses, τ, exerted by the cell on its underlying substrate, thereby controlling cell shape and lineage commitment. It is further object to control cell strain, which further controls cell shape and differentiation, such that there is an inverse relationship between intracellular and extracellular strains. On stiff matrices, cell strains are large, while matrix strains are small, and on soft matrices, cell strains are small, while matrix strains are large.

It is also an object to provide a method for regulating differentiation and cell shape of an anchorage-dependent cell by varying the elasticity of a biocompatible substrate, e.g., an underlying hyaluronic acid (HA) substrate. Moreover, it is an object to provide methods for finely tuning the HA elasticity, e.g., from about 0.1 kPa to about 150 kPa though the use of molecular weight, HA concentration, cross-link density, timepoint of inactivation, or combinations thereof.

Additional objects, advantages and novel features of the invention will be set forth in part in the description, examples and figures which follow, all of which are intended to be for illustrative purposes only, and not intended in any way to limit the invention, and in part will become apparent to those skilled in the art on examination of the following, or may be learned by practice of the invention.

### BRIEF DESCRIPTION OF THE FIGURES

The foregoing summary, as well as the following detailed description of the invention, will be better understood when read in conjunction with the appended drawings in which like numerals designate like elements. It should be understood, however, that the invention is not limited to the precise arrangements and instrumentalities shown.

FIGS. 1A-1B show results for mesenchymal stem cell differentiation as a result of tissue elasticity. FIG. 1A graphically shows that solid tissues exhibit a range of elastic moduli, E. FIG. 1B schematically illustrates a mesenchymal stem cell (MSC) and the environment used in the in vitro gel system of the present invention, allowing for a tunable elastic modulus through changes in cross-link density, for control of cell adhesion by covalent attachment of collagen-I, and for control of thickness h by adjusting the volume of polymerizing solution. Also shown in FIG. 1B presents MSC images corresponding to different substrate elasticity (0.1-1 kPa, 8-17 kPa and 30-40 kPa) and different times (4 and 96 hours). The scale bar is 20 μm. Graphs inset in FIG. 1B quantify morphological changes versus E. Graph (i) shows cell branching per length of primary E13.5 mouse neurons, MSCs, and blebbistatin-treated MSCs and graph (ii) shows spindle morphology of MSCs, blebbistatin-treated MSCs, and mitomycin C-treated MSCs (open squares) compared to C2C12 myoblasts (dashed line).

FIGS. 2A-2B show that matrix stiffness alters the adhesion-contractile balance. FIG. 2A shows that focal adhesion size as a percentage of cell area increases with matrix elasticity. The inset shows focal adhesion distributions scale with focal adhesion size, and shift to larger adhesions for higher stiffness substrates. FIG. 2B shows contractility or cell pre-stress, σ (the net tensile force over the cell-matrix interface carried by the actin cytoskeleton across a cross-sectional area of the cell that balances cell traction forces), in both MSCs and control cell lines increases linearly with substrate elasticity, E. The inset image shows a myoblast (outlined) displacing beads embedded in the gel (black arrows) that equates to a strain field represented by the color map (white is high strain). The scale bar is 10 μm. In the lower plot of FIG. 2B, membrane cortical stiffness (measured by micropipette aspiration) increases with gel stiffness, but blebbistatin treatment softens all cell membranes more than 3-fold. The middle inset of FIG. 2B shows mean intracellular strain, εᵢₙ, versus the mean extracellular strain, εₒᵤₜ, fit to a power-law (εᵢₙ=B*εₒᵤₜ^{b}) for all cell types.

FIG. 3 shows cell spreading differences between cells grown on an ultra-thin polyacrylamide gel, <500 nm, and a polyacrylamide gel of normal thickness, i.e., ˜70 microns. As shown, cell spreading on thin gels can be mapped to that of thick gels to determine an ‘apparent’ gel modulus for cells on these thinner materials (inset graph). Dark gray shaded region represents Eₐₚₚₐᵣₑₙₜ, given experimental uncertainties.

FIGS. 4A-4B show how the cell membrane's cortical stiffness in FIG. 7B was measured. FIG. 9A shows the micropipette schematic for membrane aspiration. FIG. 9B shows sample aspiration data of 5 cells from a single matrix stiffness fit by Equation 3, showing the range of variability. The inset images illustrate this phenomenon, with arrows indicating the membrane cap. The scale bar is 5 μm.

FIG. 5 summarizes the observed elasticity-coupled lineage commitment for a pluripotent mesenchymal stem cell.

FIG. 6 depicts chemical modification of HA by thiol group cross-linking. In the reaction scheme of the thiol addition, PDPH is coupled to the carboxyl group of HA using carbodiimide chemistry at pH 4.75 with subsequent reduction of the disulfide bond of PDPH using dithiothreitol (DTT) at pH 8.5 to yield the free thiol group.

FIG. 7 depicts a graph of deflection (d) v. sample height (z), representative curve of a gel with a Young's modulus E=11 kPa. Black line shows collected data points and broken line denotes the best fit using a modified Hertz model

FIG. 8 shows the effective cell adhesion and morphology MSCs of on elastically-modified PA and HA substrates. MSCs adhere and spread well on PA and HA gels and exhibit similar morphology.

FIGS. 9A-9D show the tunable elasticity E of the matrix dictates cell morphology independent of material. FIG. 9A graphically shows Young's elastic modulus E of HA gels measured by AFM (black open squares) scales linearly (solid line denotes a linear fit with R²=0.999) with the amount of cross-linker up to a critical cross-linker/thiol ratio of 0.5 beyond which the elasticity decrease due to dangling cross-linkers. FIG. 9B graphically shows that Young's modulus E increases with HA-S concentration and is fitted by a scaling law (solid line) as expected for crosslinked polymer networks. The circle with a cross is the shear modulus G′ measured with a cone and plate rheometer that was converted to the respective Young's modulus value assuming a Poisson ratio of v=0.45. FIG. 9C depicts collagen dependent adhesion of hMSCs. Graph showing the spread cell area A on HA (open squares) and PA gels (open circles) increased with increasing surface concentration of collagen I fitted with the sigmoidal function in the inset (broken lines) yielding similar affinity constants k for HA and PA gels (error bars represent SEM). The two images show cells representing the average values for the lowest concentration and the saturation regime. The inset on the lower right shows no significant difference in spread cell area between HA and PA gels pre-incubated with serum and the PBS controls. FIG. D shows fluorescence images (top) of cells 24 h after plating on collagen-coated HA gels of different elasticity (E=1, 11, and 34 kPa) best representing the average values. Scale bar represents 100 μm. Bottom left plot shows spread cell area A on HA (open squares) and PA (open circles) substrates of varying elasticity and glass (gray closed square) fitted with the equation in the inset yielding similar values for k (error bars are SEM). Bottom right plot shows the spindle factor r shows a peak at 11 kPa for both hydrogel types showing isotropic cell shapes on very soft and very rigid substrates but highly elongated cells on the intermediate stiffness. The dashed line is the best fit using a chemo-mechanical model.

FIG. 10A-10C show conformal 3D matrices. FIG. 10A is a drawing showing the sandwich overlay procedure. Cells were plated on a base hydrogel of elasticity E₀ and after time t (1 hour or 24 hours) the second hydrogel of stiffness E₁ was added creating a fully conformal overlay. FIG. 10B graphically shows the spindle factor r of the cells is dictated by matrix elasticity and the additional dimensionality in terms of conformal overlay amplifies the impact. FIG. 10C shows the spread cell area A for the different sample geometries indicating that on 2D substrates the spreading process is nearly complete after 4 hours, and that the overlay on the soft substrates (E₀=1 kPa) barely affects cell area, but the additional layer on the stiff substrates (E₀=11 kPa) leads to an area decrease.

FIG. 11 shows that matrix elasticity also dictates nucleus morphology, see solid line is a best fit of a sigmoidal function and the dashed line shows the respective value for cells encapsulated in 3D matrices, and is the fit of FIG. 9D rescaled to illustrate the observed that the data follows the trend for the overall cell shape. See data point showing nuclei of cells on 11 kPa substrate treated with blebbistatin.

FIG. 12 graphically shows that quenching yields stable gels. There is shown a significant increase of gel stiffness with time after day 1, as shown by the open squares, as remaining free thiol groups form disulfide bridges and introduce additional crosslinks. To avoid continuous stiffening, further auto-crosslinking was achieved by treating the gels with a 1.5% solution of iodacetamide in PBS (or 2% cysteine in PBS) for two hours to cap the thiol groups, after which the mechanical properties remained stable as depicted by the closed circles.

### DETAILED DESCRIPTION OF CERTAIN PREFERRED EMBODIMENTS

The present invention provides a method for regulating the differentiation of mesenchymal stem cells (“MSCs”) in response to tissue elasticity, particularly while maintaining biocompatibility. In some aspects of the invention, cell morphology shows that lineage commitment is influenced by matrix stiffness (elasticity).

Regardless of geometry, the intrinsic resistance of a solid to a stress is measured by the solid's elastic (or Young's) modulus E, which is most simply obtained by applying a force, such as hanging a weight, to a section of tissue or other material and then measuring the relative change in length or strain. Another common method to obtain E involves controlled macro- or micro-indentation, including atomic force microscopy (AFM). The elastic modulus E is discussed, e.g., by Sugawara et al., Hearing Research 192:57-64 (2004); Taylor et al. J. Biomech. 37:1263-1269 (2004); Engler et al., 2004c, supra. Many tissues and biomaterials exhibit a relatively linear stress versus strain relation up to small strains of about 10 to 20%. The slope E of stress versus strain is relatively constant at the small strains exerted by cells (Lo et al., 2000, supra), although stiffening (increased E) at higher strains is the norm (Storm et al., Nature 435:191 (2005); Fung, A First Course in Continuum Mechanics: For Physical and Biological Engineers and Scientists (Prentice Hall, Englewood Cliffs, N.J., ed. 3, 1994).

Nonetheless, microscopic views of both natural and synthetic matrices, e.g., collagen fibrils and polymer-based mimetics (Stevens et al., Science 310:1135 (2005)), suggest that there are many subtleties to tissue mechanics, particularly concerning the length and time scales of greatest relevance to cell sensing. The elastic resistance that a cell ‘feels’ when it attaches to a substrate is governed by the elastic constant E of the substrate or tissue microenvironment. Sample preparation is also critical. For example, macroscopic elastic moduli measurements of whole brain can vary 2-fold or more, depending on sample preparation, perfusion, etc. (Gefen et al., J. Biomech. 37:1339 (2004)). In addition, many single or multi-cell probing methods involve high-frequency stressing (Hu et al., Am. J. Physiol. Cell Physiol. 287:C1184 (2004)), whereas relevant time scales for cell-exerted strains seem likely to range from seconds to hours, motivating long time studies of cell rheology (Bao et al., Nat. Mater. 2:715 (2003); Wottawah et al., Phys. Rev. Lett. 94:098103 (2005)). Regardless, comparisons of E (in units of Pascal; “Pa”) of three diverse tissues that contain a number of different cell types show that brain tissue is softer than muscle (skeletal muscle) (Engler et al., 2004c, supra; Yoshikawa et al., Biochem. Biophys. Res. Commun. 256:13 (1999)), and muscle is softer than collagenous bone (Engler et al., 2004c, supra; Taylor et al., J. Biomech. 37:1263-1269 (2004)). Although mapping soft tissue micro-elasticities at a resolution typical in histology is important, the implication here is that there are distinct elastic microenvironments for epithelial cells and fibroblasts in skin, for myotubes in fiber bundles, for neurons in brain, etc.

Correlations have long been made between increased cell adhesion and increased cell contractility (e.g., Leader et al., J. Cell Sci. 64:1 (1983)), but it now seems clear that tactile sensing of substrate stiffness feeds back on adhesion and cytoskeleton, as well as on net contractile forces, for many cell types. Seminal studies on epithelial cells and fibroblasts exploited inert polyacrylamide gels with a thin coating of covalently attached collagen (Pelham et al., 1997, supra). This adhesive ligand allows the cells to attach and, by controlling the extent of polymer cross-linking in the gels, E can be adjusted over several orders of magnitude, from extremely soft to stiff.

Because tissue elasticity is a factor in MSC differentiation, matrix elasticity is mimicked in vitro in the present invention with relatively inert, collagen I-coated polyacrylamide gels in which the concentration of bis-acrylamide cross-linking sets the elasticity (Pelham et al., 1997, supra). Solid phase gels for 2-dimensional electrophoresis generally are made of a porous polymer, such as polyacrylamide, or of hyaluronic acid, and are constructed using known methods. To minimize variability, it is beneficial if the materials and methods for making the gels are reproducible (see, e.g., the Examples that follow), and perhaps, produced by an automated means to reduce introduced variability. Gel monomers are mixed with agents that induce polymerization and then are poured or layered into a mold that dictates the size and shape of the polymerized gel. For example, the catalyzed liquid gel monomer can be poured between glass plates separated uniformly over the entire surfaces thereof to produce a square or rectangular slab gel. The glass plates, separated by about a millimeter or a fraction thereof, are held in place until the gel is formed. The concentrations of polyacrylamide gels used in electrophoresis are generally stated in terms of % T (the total percentage of acrylamide in the gel by weight) and % C (the proportion of the total acrylamide that is accounted for by the cross-linker used). N,N′-methylenebisacrylamide (“bis”) is typically used as a cross-linker. Layered gels are disclosed in greater detail in the Example section.

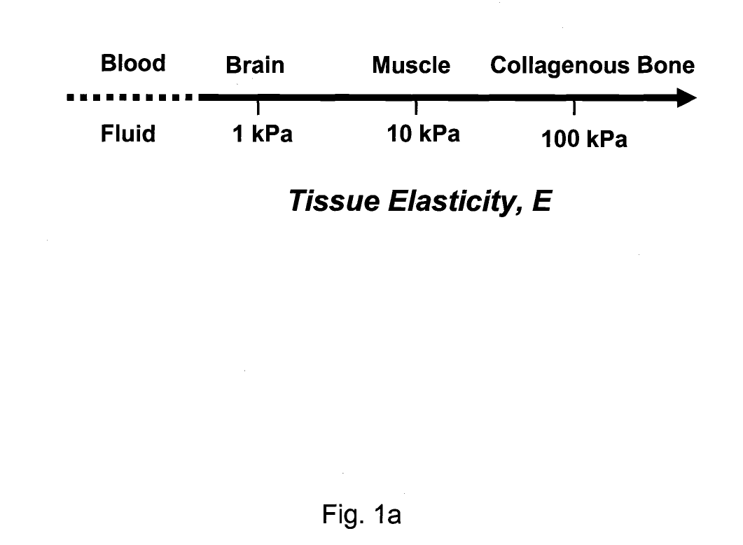

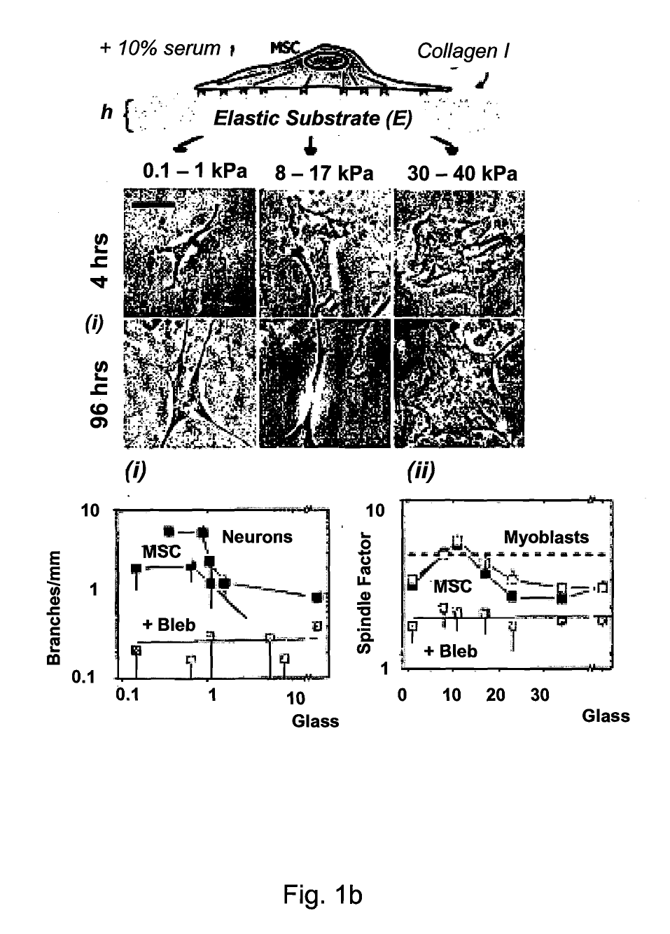

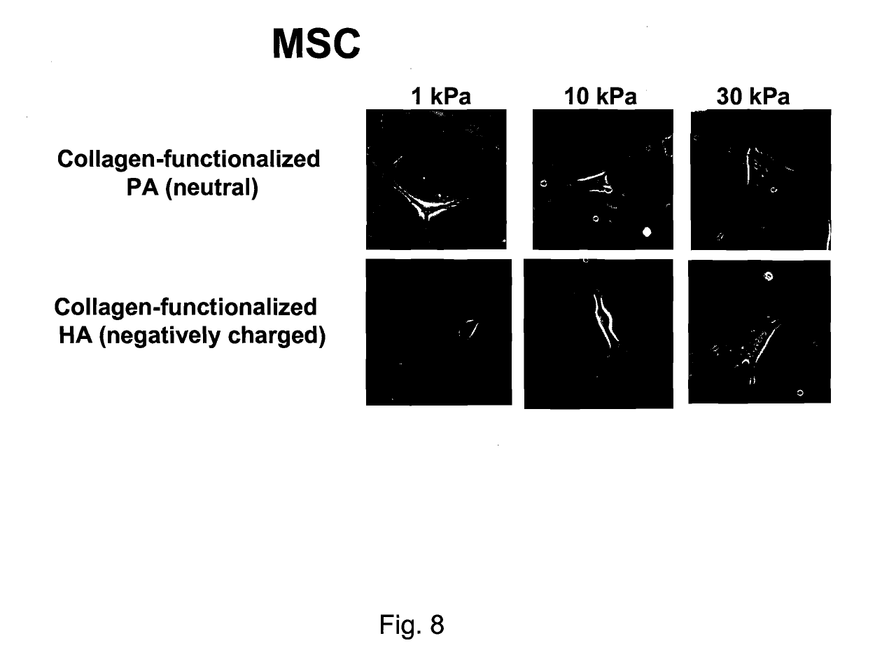

Using these tunable gel systems and sparse cultures, FIGS. 1A-1B show exemplary results for MSC differentiation as a result of tissue elasticity. This is further apparent in FIG. 8 on a hyaluronic gel system. For preparation, see e.g., the Examples that follow. A stable population of MSCs was biased towards developing different lineages, i.e., neurons, myoblasts, and osteoblasts—all in identical serum conditions and all on collagen-I (see, e.g., FIG. 1B), thereby supporting differentiation into these three phenotypes (Engler et al., 2004c, supra; Garcia et al., J. Dent. Res. 84:407-413 (2005); Stephansson et al., Biomaterials 23:2527-2534 (2002); Yoneno et al., J. Biomed. Mater. Res. A 75(3):733-741 (2005)). In support of differentiation, the outside-in, E-regulation of key lineage markers and myosins, as well as activators/effectors, such as Rho that cross-correlates with lineage-specific transcription factors and metrics of contractility, were monitored and documented (see Examples).

Further provided is a specific, and biocompatible hydrogel system based on a thiol-modified hyaluronic acid (HA-S) that meets all of the requirements outlined above: its Young's modulus E can be well-controlled by variation of the concentration of cross-linker or HA-S. However, an HA hydrogel can be polymerized without toxicity to the cells, allowing for 3D cell culture either by encapsulation of the cells during the polymerization process or using a ‘sandwich’ technique, where the cells are cultured on a 2D substrate and are subsequently covered with the HA-S hydrogel that forms a conformal overlay. Although earlier reports of the mechanical properties of similar HA-S hydrogels also show a crosslinker concentration dependent elasticity, they failed to achieve the wide range of elasticities needed to encompass the physiological range (Ghosh et al., Biomacromolecules, 6(5):2857-2865 (2005)), nor did they exhibit a finely tunable elastic modulus within the physiologically relevant range Burdick et al., Biomacromolecules 6(1):386-391 (2005); Masters et al., Biomacromolecules 26(15):2517-2525 (2005)).

By adapting the disclosed protocol for thiol modification of HA, employing a monofunctional addition group instead of the disulfide-containing dihydrazides, and by using a higher molecular weight HA (MW=413 kDa), as well as a higher crosslinker density, it was possible to achieve a finely tunable Young's elastic modulus E from 0.1 to 150 kPa, covering the whole physiologically relevant range. This offers a biocompatible hydrogel as a culture matrix for human mesenchymal stem cells (hMSCs) and presents a sandwich system that yields a fully conformal overlay of cells, demonstrating that the cytoskeletal arrangement is dictated by matrix elasticity in both two and three dimensions.

In an alternative embodiment of the present invention, MSC differentiation is blocked or inhibited with an inhibitor of non-muscle myosins (NMM II), blebbistatin. Nevertheless, as demonstrated in the Examples section that follows, soluble inductive factors tend to be less selective than matrix stiffness in stimulating differentiation. Moreover, by controlling gel thickness h, the distance between a MSC and its substrate (that influences differentiation) was determined, and physically defined the microenvironment surrounding the MSCs.

As graphically shown in FIG. 1A, solid tissues exhibit a range of elasticity, as measured by the elastic modulus, E (the ratio of stress to strain, providing a measure of the stiffness of a material), ranging from less than 1 kPa to more than 100 kPa, depending on cell type. For example, E values of about 1 kPa generally correspond to brain tissue, E values about 10 kPa generally correspond to muscle tissue and E values about 100 kPa generally correspond to collagenous tissue.

### Effect of Cell Morphology: Lineage Commitment Influenced by Matrix Stiffness

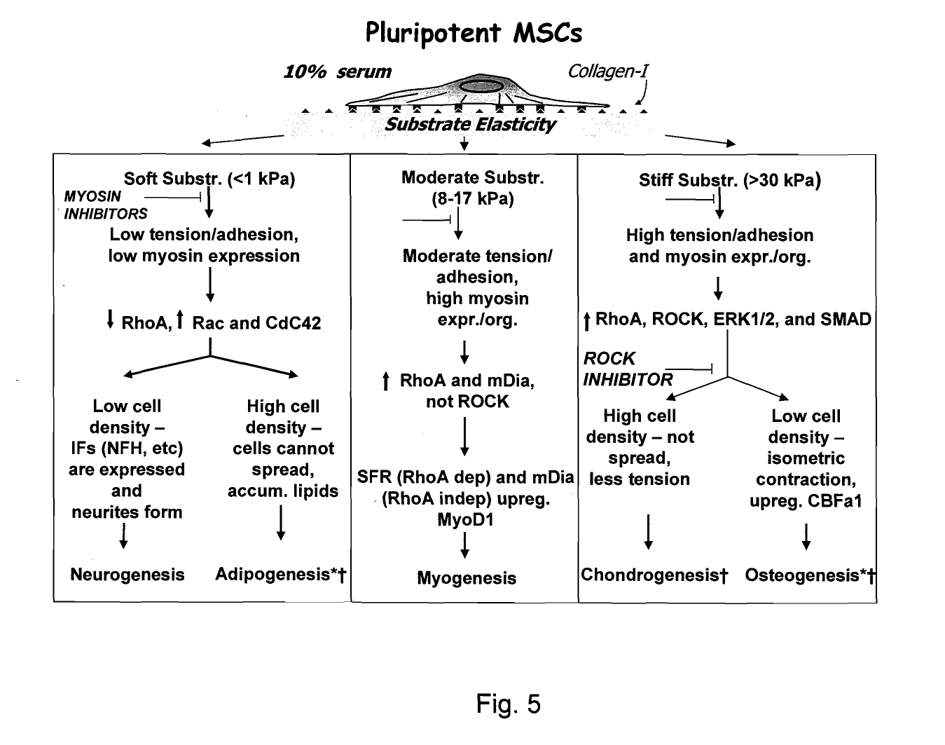

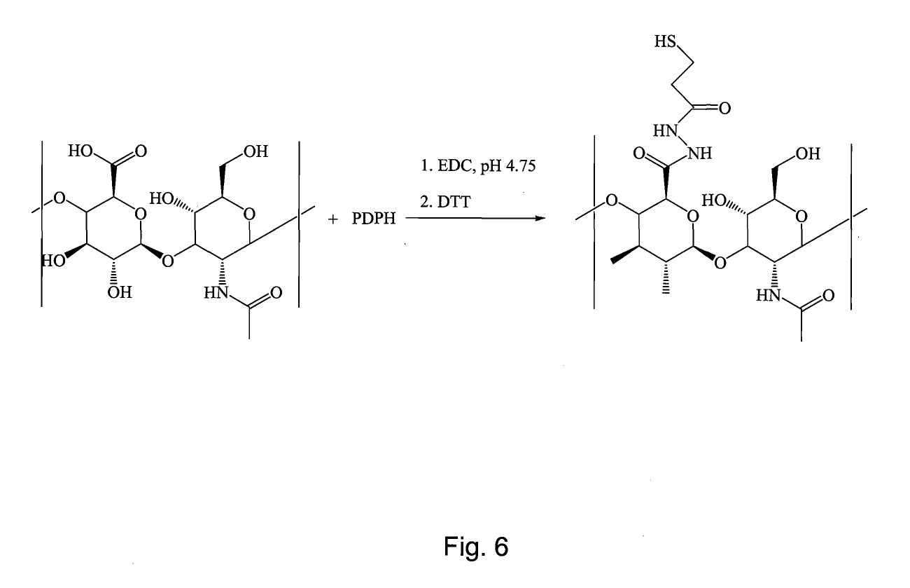

In the in vitro gel system, on soft, collagen-coated gels that mimic the elasticity of brain tissue (E_{brain}≈0.1-1 kPa), the vast majority of MSCs exhibit a branched morphology (schematically shown in FIG. 1B). This gel system allowed for the control of E through cross-linking, for control of cell adhesion by covalent attachment of collagen-I, and for control of thickness h. The MSC images in FIG. 1B and in FIG. 5 correspond to different substrate elasticities (0.1-1 kPa, 8-17 kPa and 30-40 kPa) at 4 hours and 96 hours. MSCs have a neurite-like branched shape, myoblast-like spindle shape, or osteoblast-like polygonal morphology when cultured on gels in the range of either ˜E_{brain} (0.1-1 kPa), ˜E_{muscle} (8-17 kPa), or the stiffest, collagenous bone-like gels (30-40 kPa), respectively. The branching densities of MSCs approached those of primary neurons on matrigel-coated gels (Flanagan et al., 2000, supra).

In contrast, on 10-fold stiffer substrates that mimic the elasticity of striated muscle (E_{muscle}≈8-17 kPa), MSCs develop myoblast-like, spindle shapes as shown in FIG. 5. Considerably stiffer substrates (30-40 kPa), that reasonably mimic collagenous bone, yield polygonal MSCs that are similar in shape to osteoblasts. A quantitative analysis of the cell shapes (FIG. 1B, graphs (i) and (ii)) shows that variations in morphology are about the same for MSCs as they are for differentiated cells. Graph (i) shows cell branching per length of primary E13.5 mouse neurons, MSCs, and blebbistatin-treated MSCs; whereas graph (ii) shows spindle morphology of MSCs, blebbistatin-treated MSCs, and mitomycin C-treated MSCs, as compared to C2C12 myoblasts. Changes in cell shape (<4 days), especially the development of neurite-like branches or spindle-like morphologies, were quantified either by the number of membrane branches per mm of cell, or by a “spindle factor,” the major cell axis/minor cell axis, respectively (see Examples). Furthermore, since the inhibition of proliferation by mitomycin-C (FIG. 1B(i), open squares) has little impact on average cell shape, the morphological results provided herein are consistent with lineage development being a population-level response to substrate elasticity.

For cells on any substrate, blebbistatin was found to block branching, elongation, and any significant spreading of MSCs (FIG. 1B, plots(i) and (ii)). While less specific myosin inhibitors, such as BDM (at mM concentrations), are already known to block neuronal motility (Ruchhoeft et al., J. Neurobiol. 32:567-578 (1997)), as well as the sensitivity of differentiated cells to substrate elasticity (Pelham et al., 1997, supra), blebbistatin appears to be a more selective and potent inhibitor (Straight et al., Science 299:1743-1747 (2003)). For example, blebbistatin inhibits NMM II ATPase activity (at <10 μM drug concentrations) without affecting myosin light chain kinase (“MLCK”), as well as blocking cell blebbing and rapidly disrupting directed cell migration and cytokinesis in vertebrate cells. Myosin crystal structures show that the Mg ATPase binding pocket for blebbistatin relies on the small alanine side chains that are unique to NMM II, isoforms A, B, C, and myosin-VI, which is consistent with the most recent assays of the specificity of blebbistatin (Limouze et al., J. Muscle Res. Cell Motil. 25:337-341 (2004); Straight et al., 2003, supra). Consequently, because MSCs express the three NMM IIs, but no significant myosin-VI, as shown in the present invention, the three NMM IIs and cytoskeleton were strongly implicated in differentiation.

### RNA Profiles: Lineage Commitment on Matrices of Tissue-like Stiffness

In an embodiment of the invention, RNA profiles indicated lineage commitment on matrices of tissue-like stiffness. Transcriptional profiles of early neurogenic, myogenic, and osteogenic markers were consistent with lineage identifications based above on morphology. With reference to early passage MSCs, cells on the softest gels (E_{brain}≈0.1-1 kPa) showed the greatest expression of early neurogenic genes. Cells were cultured on 0.1, 1, 11, and 34 kPa matrices, with the results were normalized to actin levels and then compared to expression of low passage MSCs. The selected concentrations of the matrices that were tested are exemplary and are not intended to be limiting.

Neuron-specific cytoskeletal markers such as β3-tubulin and neurofilament light chain (“NFL”), as well as adhesion proteins, such as NCAM, all contributed to an average 4-fold up-regulation of the neurogenic transcripts on the softest gels relative to expression on the other gel substrates. In contrast, MSCs grown on E_{muscle}-substrates (11 kPa) expressed 8-fold more myogenic message, with clear up-regulation of relevant transcriptional proteins, such as the Pax activators and myogenic factors (e.g., MyoD). On the stiffest gels (34 kPa), MSCs expressed 3-fold greater osteogenic message, up-regulating osteocalcin and the transcriptional factor CBFα1. See also, Engler et al., Cell 126:677-689 (2006).

Late differentiation genes, such as lineage-specific integrins (α3, α7, β1D) and morphogenetic proteins, are not yet up-regulated (as seen in FIG. 1) relative to initial MSCs, suggesting that in full differentiation, matrix elasticity is likely to couple with other factors, such as soluble factors and other non-collagenous ECM components. Indeed, differentiation marker expression, as elaborated below, appears to average about 50% of control cell levels. This commitment is also lineage specific because transcriptional profiles of early versus late MSCs (up to passage 12) do not differ significantly, even though other investigators have suggested that significant population expansion dramatically alters MSCs.

RNA levels were obtained for initially isolated MSCs (passage 4), as well as MSCs expanded in culture (up to passage 12). MSCs from these groups were plated onto 0.1, 1, 11, and 34 kPa matrices, grown for 7 days, and also profiled. Data was normalized to total actin levels and scaled from 0 (no expression) to 1 (maximal expression). Notably, there was not a dramatic RNA change between initially isolated and expanded MSCs.

### Cytoskeletal Markers and Transcription Factors: Lineage Commitment

In another embodiment of the invention, cytoskeletal markers and transcription factors can also indicate lineage commitment. When protein and transcript profiles were measured as a function of elasticity for mesenchymal stem cells, they were found to be elasticity-dependent under identical media conditions and protein markers, consistent with E-dependent expression profiling. Blebbistatin blocked all marker expression by MSCs. A majority of cells on the softest, neurogenic matrices expressed the intermediate filament protein phosphorylated neurofilament heavy chain. This protein is visible in long, branched extensions, but is poorly expressed, if at all, in cells on stiffer gels. However, when differentiation media was substituted with myoblast induction media (“MIM”) or osteoblast induction media (“OIM”), MyoD1 or CBFα1 expression occurred on all substrates, peaking near control cell expression with peaks at E*≈0.3 kPa, 10 kPa, 30 kPa and with best fit values for (K, m, and Teff): (2.8·10-4, 2.4, 9.5·10-8), (2.2·10-2, 4.8, 3.4·10-7) (4.2·10-2, 8.1, 1.3·10-6). When normalize to actin, western blots confirmed lineage commitment with matrix or soluble ligand alone, but CBFα1 and MyoD expression only reached control levels when both matrix elasticity and soluble ligand were conducive for differentiation.

Although chemical agonists (Woodbury et al., J. Neurosci. Res. 69:908-917 (2002)) reportedly induce reversible branching in MSCs, ‘branched’ fibroblasts can also be induced chemically (Neuhuber et al., J. Neurosci. Res. 77:192-204 (2004)), which suggests a pan-matrix mechanism with soluble factors. In contrast, primary fibroblasts (FC7) did not branch on the soft elastic substrates (not shown), which implies that matrix stiffness-driven neurogenesis of MSCs is specific to these pluripotent cells, as well as to committed neurons.

### Lineage Specific Activators

To better identify lineage-specific activators of key transcription factors, a cross-correlation function, Φ, was developed to compare the elasticity-dependent expression of each activator/effector gene with key transcription factor genes. These are respectively denoted as gene x(E) and gene y(E) in the function:

$$Φ = \frac{\sum ( x - \overline{x} ) ( y - \overline{y} )}{\sqrt{\sum {( x - \overline{x} )}^{2} {( y - \overline{y} )}^{2}}} \qquad Equation 1$$

A value of Φ=1 indicates that genes x and y have identical elasticity-dependence, i.e., expression between genes is similar regardless of microenvironment, whereas a value of Φ=−1 indicates an inverse correlation, i.e., gene expression trends are highly variable across different matrices.

Stem Cell Differentiation: Adhesion and Contractility Balance, but Increase with Matrix Stiffness

Like the matrix-directed variation of activators, select focal adhesion transcripts, such as non-muscle α-actinin, filamin, and talin also appear to be particularly stiffness-sensitive and driven in expression by the stiff substrates. In contrast, collagen and laminin transcripts appear relatively uninfluenced in these elastic matrix culture systems. Without wishing to be so bound by any hypothesis, this seems likely due to the fact that collagen is already attached to a matrix of specified stiffness. As a result, MSCs simply respond to the matrix, rather than remodel it. Previous work with collagen-I coated gels, indeed, shows that above a threshold level of adhesive ligand, spreading of tissue cells and their cytoskeletal organization is relatively insensitive to ligand (Engler et al., Surface Science 570:142-154 (2004b)).

Consistent with the transcription profiles for activators/effectors and adhesions, as well as the earliest reports of substrate-stiffness responses (Engler et al., 2004a, supra; Gaudet et al., Biophys. J. 85:3329-3335 (2003), stiff substrates were found to promote paxillin integration in the growth and elongation of focal adhesions. Rigidification of cell-derived three-dimensional (3D) matrices altered 3D-matrix adhesions, and the adhesions were replaced by large, nonfibrillar focal adhesions similar to those found on fixed 2D substrates of fibronectin (Cukierman et al., 2001, supra). Consistent with a role for signaling in stiffness sensing, tyrosine phosphorylation on multiple proteins (including paxillin) appears to be broadly enhanced in cells on stiffer gel substrates; whereas, pharmacologically induced, nonspecific hyperphosphorylation drives focal adhesion formation on soft materials.

Actomyosin is the contractile element of the myotubule. On very soft gels that are micropatterned with collagen strips so as to generate well-separated myotubes, actomyosin appeared diffuse after weeks in culture. However, on very stiff gels, as well as on glass micropatterns, stress fibers and strong focal adhesions predominate, suggesting a state of isometric contraction. Notably, on gels with an elasticity that approximates that of relaxed muscle bundles (E˜10 kPa), a large fraction of myotubes in culture exhibited definitive actomyosin striations. Actomyosin striation is even more prominent when cells are cultured on top of a first layer of muscle cells (as shown in Discher et al., 2005, supra). The lower myotubes attached strongly to glass and formed abundant stress fibers: whereas the upper myotubes differentiated to the more physiological, striated state. Although cell-cell contact may provide additional signals, the elasticity E of the myotubes, as measured by atomic force microscopy, was in the same range as that of gels that are optimal for differentiation, and importantly, was in the same range as that of normal muscle tissue.

Cell-cell contact appears to induce similar cell-on-gel effects for systems other than muscle. Astrocytes growing on glass, for example, appeared to provide a soft cell “stroma” adequate for neuronal branching that is similar to gels having brainlike E. Cell-cell contact may have a similar effect when cells are grown at a high density. When endothelial cells are confluent, the cells have indistinguishable morphologies on soft versus stiff substrates, whereas cells attached only to an underlying stiff surface differ in their spreading and cytoskeletal organization. Related results are also emerging from the present invention using epithelial cells and fibroblasts, as well as cardiomyocytes, showing a tendency to aggregate and form cell-cell contacts in preference to contact with soft gels.

Inhibition of actomyosin contractions largely eliminated prominent focal adhesions, whereas stimulation of contractility drives integrin aggregation into adhesions (Chrzanowska-Wodnicka et al., 1996, supra). Additionally, although microtubules have been proposed to act as “struts” in cells, and thus limit wrinkling of thin films by cells (Pletjushkina et al., Cell Motil. Cytoskeleton 48:235 (2001)), quantification of their contributions to cells on gels shows that they provide only a minor fraction of the resistance (14%) to contractile tensions. Most of a cell's tension is thus resisted by matrix (Wang et al., Proc. Natl. Acad. Sci. USA 98:7765 (2001)). On the stiffest, osteogenic gels (34 kPa), represented by thin gels of h≈0.5 μm, the adhesions were long and thin and slightly more peripheral than they appear on glass. Actin assembly followed, which generalizes the E-driven assembly of the cytoskeleton to MSCs. Consistent with this, NMM-II is already known to influence focal adhesions (Conti et al., J. Biol. Chem. 279:41263-41266 (2004)).

Materials ranging from fibrin gels and microfabricated pillars to layer-by-layer polymer assemblies (Georges et al., Appl. Physiol. 98:1547 (2005); Raeber et al., Biophys. J. 89:1374 (2005); Saez et al., in press; Wang et al, in press; Engler et al., 2004b, supra)), all suggest a similar trend of more organized cytoskeleton and larger, more stable adhesions with increasing E as outlined in the present invention, despite likely differences in adhesive ligand density and long-time elasticity. However, the responses appear to be specific to anchorage-dependent and/or relatively contractile cells.

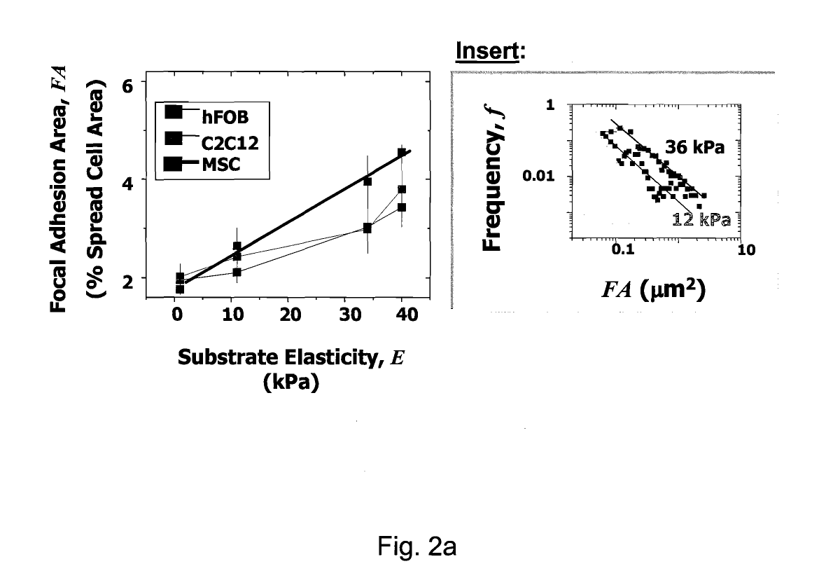

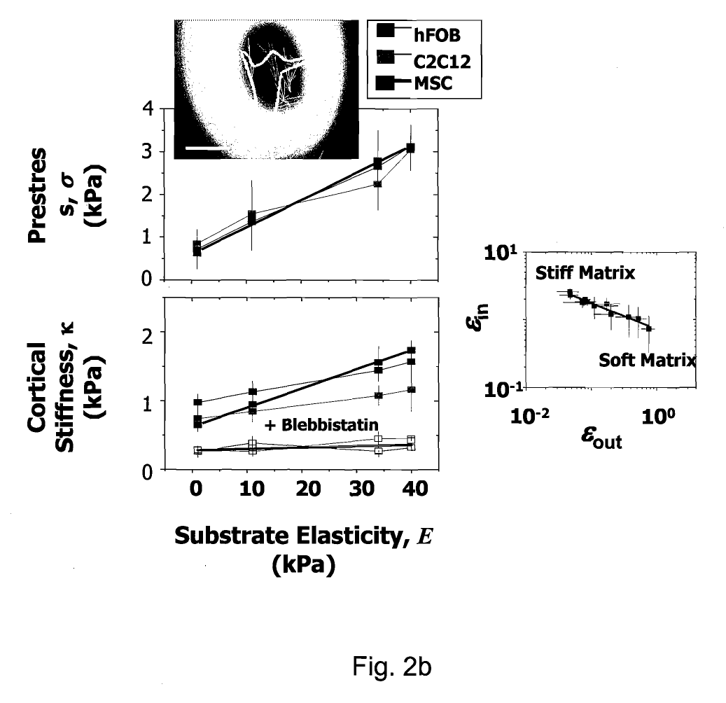

To assess adhesions and to begin addressing length scales of “micro” environments, e.g., the volume of the environment that interacts with the cell, which is on the scale of nanometers to microns, thin polyacrylamide (PA) or hyaluronic acid (HA) gels were cast with spacer beads to a thickness of h˜500 nm. This length scale allowed for low-intensity total internal reflectance fluorescence (TIRF) microscopy (Axelrod et al., J. Microsc. 129(Pt 1): 19-28 (1983)). MSCs plated on thin, but stiff, matrices spread more with many more large focal adhesions (see FIG. 2A). Yet from the viewpoint of a cell, a thin soft gel on glass is perceived as having an apparent elastic modulus greater than the gel modulus due to the proximity of the rigid coverslip. Cell adhesions, therefore, develop that are larger than they would be otherwise, which establishes just how far MSCs can feel: ˜1 μm on soft gels (<25 kPa). Accordingly, cells feel matrix strains localized to the scale of multiple adhesions, rather than the cellular scale.

Adhesions provide MSCs the necessary attachments to “feel” their microenvironment through acto-myosin contractions. Mechanically, contractility equates to a cellular pre-stress, σ, that balances the traction stresses, τ, exerted on the gel by the cell (Wang et al., Am. J. Physiol. Cell Physiol. 282:C606-616 (2002b)). Traction stresses (τ; force per area) were determined from bead displacements in the gel (see FIG. 2B (inset)) by computing forces with available algorithms (Butler et al., Am. J. Physiol. Cell Physiol. 282:C595-605 (2002); Dembo et al., Biophys. J. 76:2307-2316 (1999); Schwartz et al., Phys. Rev. Lett. 88:048102 (2002); Wang et al., 2002, supra). Although larger tractions are exerted on stiffer gels, typical tractions of (τ)˜1 kPa exceed, by orders of magnitude, the viscous fluid drag on any cell crawling in culture. In addition, mean cell tractions equate to mean gel strains that differ very little (εₒᵤₜ=(τ/E) ≈3 to 4%) between gels that differ by 2-fold in E. Consistent with nearly linear adhesion area increases with E, average σ for MSCs, C2C12-myoblasts, and hFOB-osteoblasts also show the same linear increase versus matrix stiffness, E (see FIG. 2B (top)). The trend implies larger deformation within the cell on stiffer matrices and larger deformation in the matrix on softer matrices.

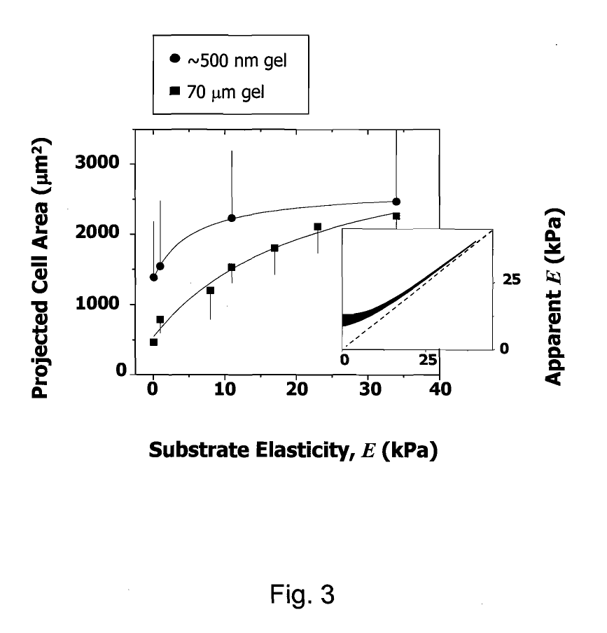

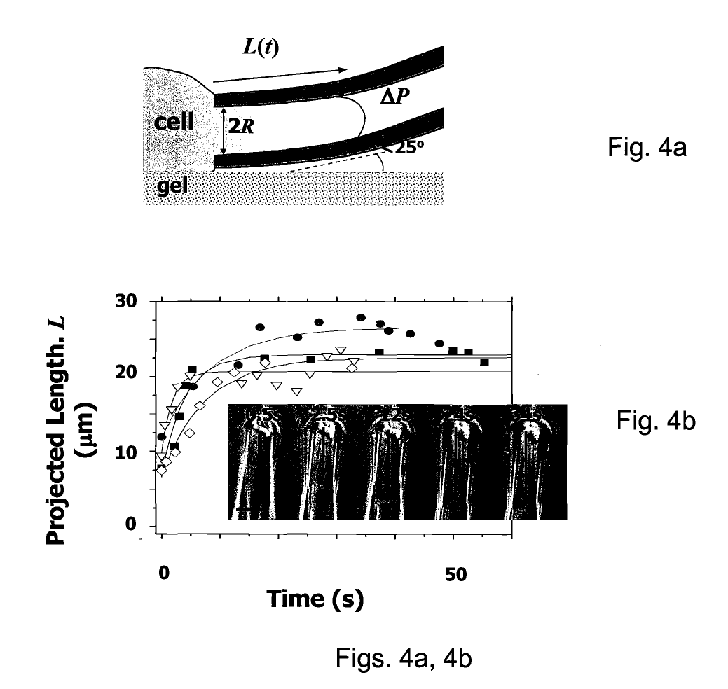

Blebbistatin, which was shown above to inhibit myosin contraction and expression, prevented any of the cells from developing either a pre-stress σ (Griffin et al., 2004, supra) or a significant cortical stiffness, κ, on any matrix (see FIG. 2B (bottom, open points)). The latter was measured by fitting a viscoelastic half-space model to membrane aspiration experiments (see FIG. 3 showing an example of cell spreading on an ultra-thin polyacrylamide gel). Cells contract their matrices up to 1-3 microns so that on thin, soft gels (h˜500 nm) attached to glass, cells are expected to ‘feel’ a matrix that is effectively stiffer than the cast gel. The result, as shown in FIG. 3, is an enhanced spreading of the cells, which allows mapping the spread area on thin gels, as compared with thick gels, and a determination of an ‘apparent’ gel modulus.

As functions of matrix stiffness, the two differentiated cell types, the C2C12-myoblasts and hFOB-osteoblasts exhibit similar (K/E) slopes, though they have distinct intercepts. By using the pre-stress results, these two differentiated cell types also show the same slope for (K/a) (0.2) as highly contractile, smooth muscle cells assayed by different techniques (Wang et al., 2002b, supra). On the other hand, MSCs appear more mechano-sensitive, with twice the slope for (κ/E) and (κ/σ). This increased mechano-sensitivity leads to a self-consistent crossover. On myogenic gels (11 kPa), MSCs and C2C12s have similar K, whereas on osteogenic gels (34 kPa), MSCs and hFOBs have similar K. Despite this difference, the inside-outside relationship between intracellular strains, εᵢₙ (=σ/κ), and the extracellular strain field, εₒᵤₜ (=τ/E), fits a universal power law for all cell types (see FIG. 2B (inset)).

One can think of such a strain comparison as similar to comparing intracellular and extracellular ion concentrations (Na⁺, K⁺, Ca⁺⁺, etc.). In the present invention, however, the inverse relationship between intracellular and extracellular strains reveals that, on stiff matrices, cell strains are large, while matrix strains are small. Whereas, by comparison, on soft matrices, cell strains are small, while matrix strains are large. The strain thus transfers from outside to in with increasing matrix stiffness, presumably activating different pathways at different strains. However, the common power law indicates a common mechanism, consistent with the central role of myosin II.

### Effective Energetics for Lineage Specification

In an embodiment of the present invention, the effective energetics for lineage specification can be determined. These findings are formalized in a simple model, wherein chemo-mechanical energetics localized to adhesions are balanced against the contractile energetics of the cell. Contractility or pre-stress, σ, is assumed to act throughout the cell volume V as a global regulator of differentiation. Coupled to this, an increase in free concentration of the local, transducing activator/effector links cooperatively to collagen (with Hill coefficient m and affinity K) and to substrate elasticity (E). The net result is a lineage commitment probability given by:

$${P}_{lineage} ( E ) = {a}_{0} + {a}_{1} exp ( \frac{- σ V}{{k}_{b} {T}_{eff}} ) [ \frac{{E}^{m}}{{E}^{m} + {K}^{m} {coll}^{m}} ] \qquad Equation 2$$

The effective thermal energy k_{b}Teff in the exponential factor should relate more to cytoskeletal stochastics than to temperature. In the limit of rigid substrates where tensions (σ) are high, such as on glass, isometric pulling on adhesions will limit differentiation of MSC. See also Engler et al., Cell supra, 2006.

Equation 2 fits three differentiation peaks (at E*≈0.3 kPa, 10 kPa, 30 kPa) with best fit values for the key parameters K, m, and T_{eff}′. (2.8·10-4, 2.4, 9.5·10-8), (2.2·10-2, 4.8, 3.4·10-7) (4.2·10-2, 8.1, 1.3·10-6). All of these parameters increased with increasing E*, and cooperativity, m, notably rises from about 2 to 8 (recall oxygen binds hemoglobin with m≈4), suggesting the progressive formation of large signaling complexes, consistent with growing adhesions.

The present invention is further described in the following Examples. These examples are provided for purposes of illustration only, and are not intended to be limiting unless otherwise specified. The various scenarios are relevant for many practical situations, and are intended to be merely exemplary to those skilled in the art. These examples are not to be construed as limiting the scope of the appended claims. Thus, the invention should in no way be construed as being limited to the following examples, but rather, should be construed to encompass any and all variations which become evident in light of the teaching provided herein.

### EXAMPLES

The following Materials and Methods were utilized in the tunable systems used to provide exemplary proofs of the principles provided in the present invention.

### Materials and Methods

Cell Culture: Human Mesenchymal Stem Cells (MSCs; Osiris Therapeutics; Baltimore, Md. or Lonza), human osteoblasts (hFOBs; ATCC, Manassas, Va.), primary human skin fibroblasts (FC7) (Engler et al., 2004c, supra), and murine myoblasts (C2C12s; ATCC) were cultured in normal growth media, such as low glucose DMEM (Invitrogen) supplemented with 10% fetal bovine serum (FBS, Sigma) and 1% penicillin/streptomycin (Invitrogen) in regular tissue culture treated flasks (Corning). See also, U.S. Ser. No. 11/351,420. Media was exchanged every other day and cells were used up to passage number nine.

For passaging of the cells, the flasks were rinsed with PBS and the cells were trypsinized with 0.25% Trypsin/EDTA (Invitrogen) for 3 minutes. After washing with media the appropriate number of cells were plated on hydrogels or replated in new flasks. To assure that cells were isolated, only 400 cells/cm² were plated on gels. To chemically induce MSC differentiation, cells were placed in the appropriate induction media, incorporated by reference from U.S. Ser. No. 11/351,420. All cells were used at low passage number, and were subconfluently cultured. Cells were plated for experiments at ˜103 cells/cm³ and cultured for 7 days, unless otherwise noted. All chemicals were purchased from Sigma (St. Louis, Mo.) unless otherwise noted.

To inhibit proliferation, cells were exposed to mitomycin C (10 μg/ml) for 2 hr and washed three times with media prior to plating. Blebbistatin (50 μM; EMD Biosciences, Inc., San Diego, Calif.), a NMM II inhibitor, was applied with every media change and was stable in culture media for up to 48 hours, as determined by thin layer chromatography.

Substrate Preparation: The water used throughout this study was purified by a Millipore system. Hyaluronic acid of different molecular weights was obtained from LifeCore (Chaska, Minn.). PDPH was purchased from Pierce. Poly(ethylene glycol) diacrylate (PEG-DA, M_{w}=3400 Da) came from Nektar Polymers (San Carlos, Calif.). All other chemicals were purchased from Sigma. PA Hydrogel Preparation. Cells were plated on variably compliant polyacrylamide (PA) gels according to a previously established protocol (Engler et al., 2004a, supra; Pelham et al., 1997, supra) herein incorporated by reference. Briefly, gel cross-linker N,N′methylene-bis-acrylamide and acrylamide monomer were varied in distilled water to achieve a polymerized solution with a tunable elastic modulus (Engler et al., 2004b, supra). Approximately 25 μl of the mixed solution was polymerized on a coverslip using 1/200 volume of 10% ammonium persulfate and 1/2000 volume of N,N,N′,N′-tetramethylethylenediamine. The polymerizing gel was covered with a dichlorodimethylsilane-pretreated coverslip to ensure easy detachment and a uniform polymerized gel surface. Final gels were 70-100-μm thick, as measured by microscopy.

To produce ultra-thin gels, however, 10 μl of a 1% polystyrene bead solution (d=250 nm; Polysciences, Inc., Warrington, Pa.) was added to polymerizing solutions, and a weight was added to the top coverslip to ensure that the gel thickness was defined by the spacer bead diameter. Type 1 collagen (0.25-1 μg/cm²; BD Biosciences, Rockville, Md.) was chemically cross-linked using a photoactivating cross-linker, sulfo-SANPAH (Pierce Biotechnology, Inc., Rockford, Ill.) and attachment was confirmed by fluorescence. Cells grown on glass coverslips (GL) alone were always coated non-specifically with collagen prior to cell seeding.

Hyaluronic Acid Hydrogel Preparation: Hyaluronic acid (M_{w}=413 kDa) obtained from Lifecore Biomedical (Chaska, Minn.) was functionalized with thiol groups by adapting the protocol of Shu et al., Biomacromolecules 3(6): 1304-1311 (2002). In brief, 3-(2-pyridyldithio)propionyl hydrazide (PDPH) (Pierce) was coupled to the carboxyl group of HA using carbodiimide chemistry. Using PDPH, a monofunctional reagent, instead of the homo-bifunctional dithiobis(propanoic dihydrazide) (DTP) avoids cross-linking of HA during the reaction that could lead to inhomogeneous modification favoring proximal intra-molecular cross-links. Then the disulfide bonds of PDPH were reduced using dithiothreitol (DTT) with subsequent dialysis (cut-off M=10 kDa) to purify the thiolated HA (HA-S) followed by lyophilization. The degree of substitution of HA-S was determined by ¹H NMR in D₂O using a 360 MHz instrument (BZH 360/52, Spectrospin and Oxford instruments). For the preparation of the hydrogels a solution of modified HA-S in DPBS was mixed with the desired concentration of cross-linker (PEG-DA). For the 3D hydrogels 100 kDa recombinant gelatin (FibroGen. San Francisco, Calif.) was modified in the same way as described for HA to yield thiol groups to be covalently bound to the hydrogel. (Gelin-S (thiol-modified gelatin) may also be used from Glycosan (Salt Lake City, Utah)).

The HA hydrogels were prepared on cover slips (25 mm diameter, Fisher Scientific) as solid support to facilitate cell culture as well as imaging and elasticity measurements. Polyacrylamide (PA) hydrogels were prepared as above as control samples, and both gel systems were coated with collagen I (BD Biosciences) using the hetero bifunctional crosslinker Sulfo-SANPAH (Pierce) as described elsewhere Pelham et al., 1997, supra; Engler et al., J. Cell Biol. 2004c, supra; Engler et al., Microtissue Elasticity: Measurements by Atomic Force Microscopy and Its Influence on Cell Differentiation, in Methods in Cell Biology, Academic Press, p. 521-545 (2007). The surface density of collagen I was determined per Engler et al., Biophys. J., 2004a, supra.

Hydrogel Sandwich Preparation: The whole procedure is performed in a biosafety cabinet to maintain sterility. All equipment used was either sterile or sterilized by UV treatment for one hour. To produce the sandwich-like structures, cells were cultured for a given time on regular 2D substrates supported by 25 mm aminosilanized cover slips, rinsed with PBS and dried of the bulk liquid. 35 μl of the hydrogel mixture was added immediately and the sample was covered with a hydrophobic chlorosilanized glass slip to create a homogenous overlay gel. After one hour the top cover slip was carefully removed and the sandwich rinsed with PBS to remove any unbound residues. Subsequently, the gels were incubated for two hours at 37° C./5.0% CO₂ in media supplemented with 1.5% or 2% cysteine (w/v) or iodacetamide solution using a shaker to cap or inactivate any remaining free thiols. Finally, the hydrogel was washed again thoroughly with PBS and/or with media and maintained at the usual cell culture conditions or stored at 4° C. until further use.

To fluorescently label the sandwich overlay, it was rinsed following gelation and then incubated while gently rocking for 2.5 min with 2 ml of a 0.4 μg/ml solution of Alexa 647 maleimide (˜1250 Da) (Invitrogen) in PBS while gently rocking for 2.5 min, followed by twice rinsing the gels with PBS.

Measuring the Young's Elastic Modulus E with Atomic Force Microscopy: Elasticity measurements on the hydrogels were performed with a MFP 1-D and a MFP 3-D atomic force microscope (Asylum Research, Santa Barbara, Calif.). Force-indentation curves were recorded using cantilevers with a pyramidal tip and a nominal spring constant k=0.06 N/m (DNP, Veeco, Santa Barbara, Calif.). The spring constant was calibrated with the thermal tune method (Hutter et al., Rev. Scientific Instruments 64(7):1868-1873 (1993)), and the elastic Young's modulus E was calculated using a modified Hertz model as reported elsewhere assuming a Poisson ratio of v=0.45 (Domke et al., Langmuir 14(12):3320-3325 (1998); Sneddon, Internatl. J. Eng Sci. 3(1):47-57 (1995)) using the following equation for a pyramidal indenter:

$$E = \frac{π ( 1 - {v}^{2} ) F}{{δ}^{2} 2 tan α} \qquad Equation 3$$

Measurements were also done using cantilevers with a sphere attached on the tip (borosilicate spheres, Novascan) with a radius R=2.5 μm leading to similar values for the elasticity using the following equation:

$$E = \frac{3 ( 1 - {v}^{2} ) F}{4 {δ}^{3 / 2} {R}^{1 / 2}} \qquad Equation 4$$

Additional measurements with an RFS II cone and plate rheometer (TA Instruments, New Castle, Del.) revealed a constant shear modulus G′ independent of the applied strain up to 25% (data not shown) in accordance with the study by Ghosh et al. Biomacromolecules 2005, supra. For the conversion of the shear modulus to the Young's modulus a Poisson's ratio of v=0.45 was assumed yielding a factor of 2.9 using the standard equation:

E=2G′(1+v) Equation 5

Immuno-staining, fluorescence microscopy, and image analysis: Cells were fixed for five minutes in a 10% solution of formaldehyde (Sigma) in PBS (Gibco) and subsequently permeabilized with a 0.5% solution of TritonX 100 (Sigma) in PBS followed by rinsing in PBS. Samples were then incubated in a 1:250 solution of the appropriate primary antibody in a 3% solution of BSA (Sigma) in PBS for at least 2 hours at room temperature on a rocker. To remove any unspecifically bound antibodies, the gels were incubated with a 0.5% solution of TritonX 100 for five minutes and rinsed with PBS twice, followed by incubation with secondary antibodies (1:250 in 3% BSA in PBS) and rhodamine-phalloidin (Sigma) for at least one hour on a rocker at room temperature. Finally, the nucleus was stained using the Hoechst dye (#33342, Invitrogen) for approximately five minutes at a dilution of 1:10,000. Again to remove all non-specific bound material the samples were incubated with a 0.5% solution of TritonX 100 for five minutes and rinsed extensively with PBS to remove residual dye for a clean background.

Fluorescence images were taken on an inverted microscope (IX 71, Olympus) using a 20× air objective (LCAch, N.A.=0.4) and a 1.6× post magnification lens, equipped with a digital Cascade 512B (Photometrix) camera, and acquired with ImagePro (Media Cybernetics). To obtain unbiased data, well isolated cells were selected in the nucleus channel and then the other fluorescence channels were recorded.

Z-stacks were recorded with a laser scanning confocal microscope (Fluoview 100, Olympus) using a 60× oil immersion objective (N.A. 1.45, Olympus). The 3D reconstruction was done with the program Voxx (Clendenon et al., Amer. J. Physiol.-Cell Physiol. 2002. 282(1):C213-C218 (2002)).

Cell area A and spindle factor r (ratio of major to minor axis of a fitted ellipse) were determined using the built-in functions of ImageJ (available at http:rsb.info.nih.gov/ij; developed by Wayne Rasband, National Institutes of Health, Bethesda, Md.). For all quantitative analysis at least 30 cells were recorded, and all cell images showed the cells to be representative of the respective average values determined by a least-square method.

### Example 1

### Differentiation Assays

1) Morphological Changes and Immunofluorescence: Changes in cell shape (<4 days), especially the development of neurite-like branches (Engler et al., 2004c, supra) or spindle-like morphologies (Flanagan et al., 2002, supra), were quantified either by the number of membrane branches per mm of cell or by a “spindle factor,” referring to the major cell axis/minor cell axis, respectively. Cells also were stained with lineage-specific antibodies: myogenesis with Myogenesis Differentiation Protein 1 (MyoD1; Chemicone International, Temecula, Calif.), osteogenesis with Core Binding Factor al (CBFα1; Alpha Diagnostic International, San Antonio, Tex.), and neurogenesis with phosphorylated and dephosphorylated Neurofilament Heavy chain (NFH; Sternberger Monoclonal, Berkeley, Calif.) along with paxillin (Chemicon), skeletal muscle myosin heavy chain (Zymed Laboratories, S. San Francisco, Calif.), and non-muscle myosin IIA and B (Sigma) or rhodamine-labeled phalloidin.

Cells were fixed with formaldehyde, incubated in a 5% albumin blocking solution for 1 hour at 37° C., permeabilized with 0.5% Triton-X-100 and incubated overnight at 4° C. in 1:100 dilution of antibodies in PBS. Cells were then incubated for 1 hour at 37° C. in 1:500 FITC-conjugated secondary and 60 μg/mL TRITC-phalloidin. Finally, cells were incubated for 10 min. in 1:100 Hoechst 33342 (Molecular Probes Europe, Leiden, Netherlands) to label DNA. Cell morphology and fluorescently labeled cells were examined on a TE300 inverted epi-fluorescent Nikon or Olympus (TIRF) microscope, imaged on a cascade CCD camera (PhotoMetrics, Huntington, Beach, Calif.), and quantified with Scion Image (Scion Corp., Frederick, Md.).

2) Western blotting: Cells (with or without blebbistatin treatment, 50 μM) were also plated on 45×50 mm coverslips to obtain enough cells for western blotting. Cells were permeablized with lysis buffer (10% SDS, 25 mM NaCl, 10 nM pepstatin, and 10 nM leupeptin in distilled water), boiled for 10 minutes, placed in a reducing SDS-PAGE gel (Invitrogen, Carlsbad, Calif.) with MOPS buffer, and run against a colorimetric molecular weight marker. Proteins were transferred onto nitrocellulose and blocked in a solution of 1% albumin, 50 mM Tris Buffered Saline (TBS). Membranes were rinsed 2× in 1% Tween 20-TBS (TTBS), and then a 1:500 solution of primary antibodies were added for 2 hours. Membranes were rinsed 2× with TTBS again and 1:1000 of the secondary HRP-conjugated antibodies were added. Color development was achieved with a HRP development kit (Bio-Rad Laboratories, Hercules, Calif.). All westerns were run in duplicate, along with an addition blot for actin and Commassie blue-staining to ensure constant protein load among samples. Quantification of western blots was done by Scion Image software.

3) Oligonucleotide Array Assays: Total RNA (3-5 μg) was obtained from MSCs (with or without blebbistatin treatment) cultured on gel substrates of varying stiffness, as well as C2C12 myoblasts on 11 kPa gels and HFOB osteoblasts on 34 kPa gels, using an ethanol-spin column extraction. The samples were labeled with an Ampolabeling Linear Polymerase Reaction kit (SuperArray Bioscience, Frederick, Md.) and hybridized to custom oligonucleotide arrays. Membranes for control (C2C12, HFOB, and MSCs from flasks), experimental (MSCs on gels), and duplicate sample RNAs were processed in parallel to reduce technical variability. Chemiluminescent signals were detected on Biomax Film (Kodak) and analyzed with Scion Image software. Background-corrected signals were normalized to a control gene, β-actin.

Creep-test Micropipette Aspiration: Micropipettes were forged using a deFonbrune-type microforge (Vibratome, St. Louis, Mo.) to a radius of 2-3 μm with an approximately 25° pipette curvature so when mounted in micromanipulators (Nirishige; Japan) at an angle similar to the micropipette's curvature. The end of the pipette was flush with the cell edge. A step pressure drop was imposed on the cell membrane, causing the membrane projection, L, to aspirate into the pipette as a function of time (t), pressure drop (ΔP) and pipette radius (R), as governed by the Sato and coworkers' viscoelastic half-space model (Sato et al., J. Biomech. Eng. 112:263-268 (1990), herein incorporated by reference:

$$L ( t ) = \frac{3 R Δ P}{π κ} [ 1 - \frac{{μ}^{′}}{κ + μ} exp ( \frac{- t}{τ} ) ] \qquad Equation 6$$

Images of the projection length were taken every 2 seconds in brightfield on a TE300 inverted Nikon microscope and cascade CCD camera (Photometrics), which allowed accurate fitting of the parameters κ, μ′, and τ to the data to determine the elastic and viscous moduli of each membrane.

Traction Force Measurements: Adhesive stresses, imposed on the matrix surface by an adherent cell, generate a displacement field from embedded beads within a soft substratum, which can be mapped on the cell if the gel can be approximated as a semi-infinite solid. Briefly, the well characterized traction force method (Butler et al., 2002, supra; Dembo et al., 1999, supra; Wang et al., 2002b, supra) uses bead displacements between images with, and without, the adherent cell to assemble a displacement field and determine Green's strain function given known material properties of the substratum (elastic modulus, Poisson's ratio, etc). The traction field was used to obtain the cell pre-stress, i.e., the net tensile force over the cell-matrix interface carried by the actin cytoskeleton across a cross-sectional area of the cell that balances cell traction forces (Wang et al., 2002b, supra).

Accordingly, while stem cell differentiation by soluble stimuli is known, the strong influence of an immobilized microenvironment on cell differentiation is provided by the present invention. The microenvironment surrounding MSCs plays a critical role in lineage commitment by influencing cell adhesion and contractility via mechano-sensitive machinery, e.g., Rho GTPases. This reorganized myosin to balance contractile forces against the sensed resistance which helped to localize and activate lineage-specific transcriptional programs.

### Example 2

### The Stiffness Model

Although the following model may be far too simplistic to capture the complexities of stiffness-controlled gene regulation, the model is inspired by the atomistic complexity of cooperative release of oxygen under hydrostatic tension (Carey et al., J. Biol. Chem. 252:4102-4107 (1977)). The goal of the following minimal model was to fit the differentiation results with the simplest formalism to incorporate the most essential physico-chemical ingredients. Two key states were assumed for the limiting association of a key, lineage-specific component, “Xᵢ.” This factor associates with apparent affinity K in or near the focal adhesions and obeys a molecular partition function (4), that cooperatively links to collagen (coil) with a Hill coefficient (ξ) to give:

ξ=1+[(K/E)coll]^{m} Equation 7

In terms of energetics, K˜exp(-ΔG/k_{b}T) and the matrix modulus E˜A exp(κx²/k_{b}T). Additionally, K is the relevant stiffness of matrix/membrane/adhesions, and x is a strain. If κx² is small, E˜A[1+(κx²/k_{b}T)] which implies that K is linear in E, as shown in FIG. 2B for cortical stiffness.

It was assumed that the fraction of unbound Xᵢ matters most for lineage specificity: θ′=1-[∂ ln(ξ)/ln(coll)]=1/ξ. This is the free and diffusible fraction of Xᵢ (not associated with collagen) that has the strongest effect. With N as the total number of species Xᵢ, the total unbound portion of this species is Θ=N θ′=N/ξ, which gives a chemo-mechanical potential for N=constant as:

G_{chem}=−k_{b}T ln(N/ξ)=constant−k_{b}T ln [1+[(K/E)coll]^{m}]⁻¹ Equation 8

The Total Free Energy depends additionally on the global pre-stress (σ), acting on the cell volume, V, Gₜₒₜ=G_{chem}+σV, which gives the lineage commitment probability (Equation 8) by taking the exponential of Gₜₒₜ.

### Example 3

### Tunable Mechanical Properties of a HA Hydrogel

A hydrogel system was developed based on a thiol-modified hyaluronic acid (HA-S) that meets all of the requirements outlined above: its Young's modulus E can be well-controlled by variation of the concentration of cross-linker or HA-S. This hydrogel can be polymerized without toxicity to the cells, allowing for 3D cell culture either by encapsulation of the cells during the polymerization process or using a ‘sandwich’ technique, where the cells are cultured on a 2D substrate and are subsequently covered with the HA-S hydrogel that forms a conformal overlay. Earlier reports of the mechanical properties of similar HA-S hydrogels also show a crosslinker concentration dependent elasticity but did not achieve the wide range of elasticities needed to encompass the physiological range (Ghosh et al., 2005, supra). While there are other reports on HA derived hydrogel systems, none of them exhibits a finely tunable elastic modulus within the physiologically relevant range. By adapting the protocol of the Prestwich group (Shu et al., 2002, supra; Cai et al., Biomaterials 26(30):6054-6067 (2005); Liu et al., Biomaterials. 26(23):4737-4746 (2005); Shu et al., Biomaterials 25(7-8):1339-1348 (2004)) for thiol modification of HA employing a monofunctional addition group instead of the disulfide-containing dihydrazides and using a higher molecular weight HA (M_{w}=413 kDa), as well as a higher crosslinker density, we achieve a finely tunable Young's elastic modulus E from 0.1 to 150 kPa, covering the whole physiologically relevant range. In the following example is demonstrated the applicability of our hydrogel as a culture matrix for human mesenchymal stem cells (hMSCs) and present a sandwich system that yields a fully conformal overlay of cells demonstrating that the cytoskeletal arrangement is dictated by matrix elasticity in two and three dimensions.

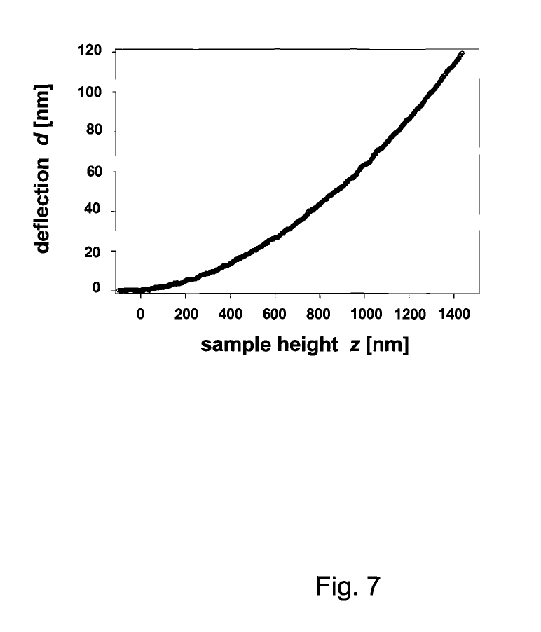

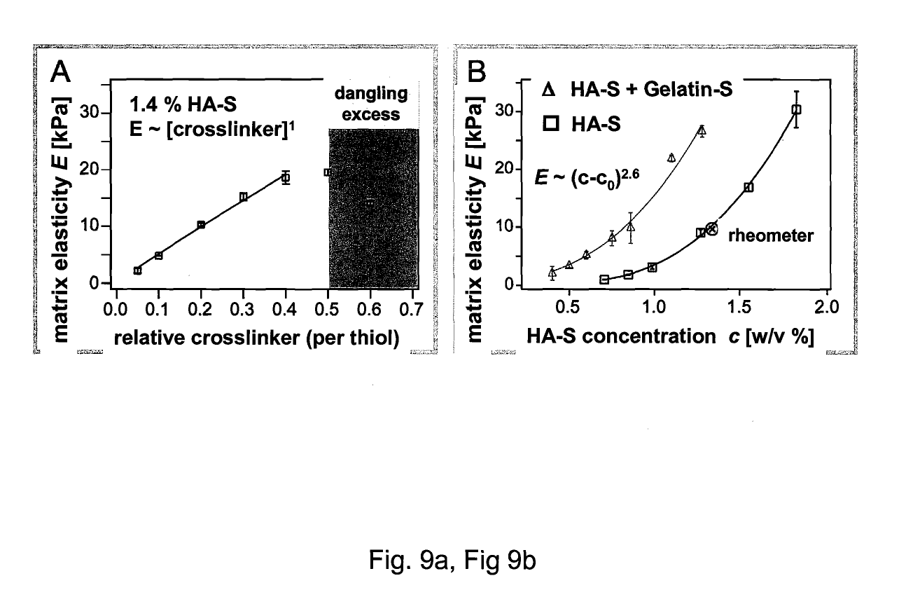

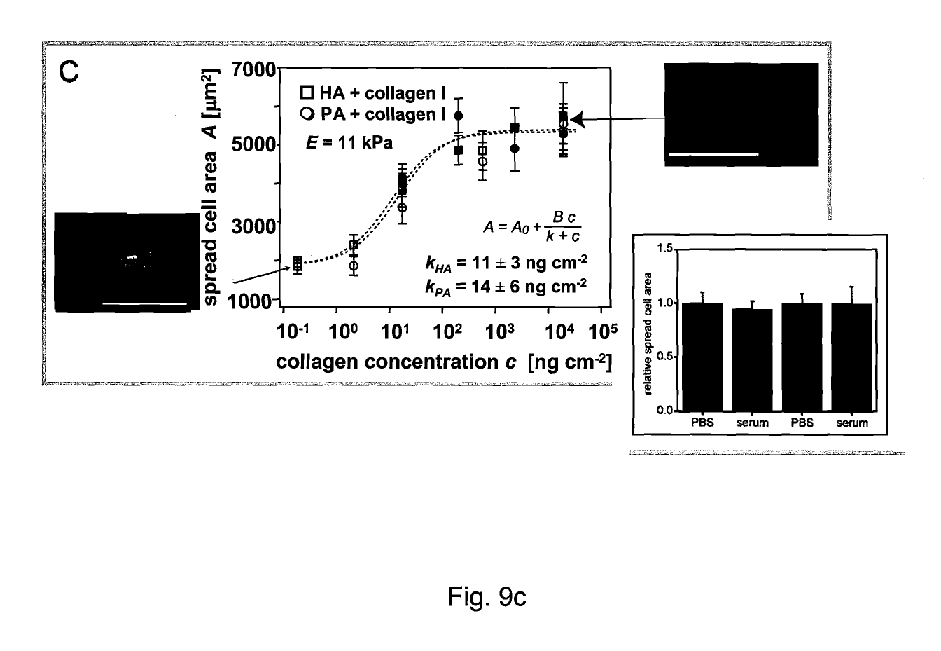

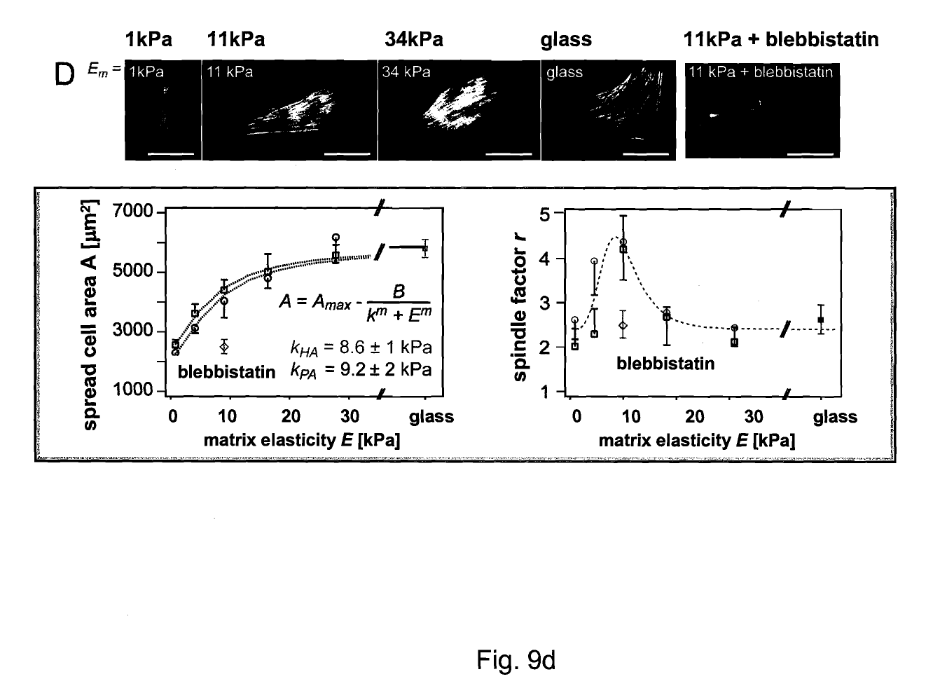

Finely Tuning Young's Elastic Modulus E: Thiol modified HA (HA-S) was prepared using a modified protocol established Shu et al., 2002, supra as described in the Materials section with a degree of substitution d.s.=0.76 as determined by ¹H NMR. Mixing HA-S with polyethyleneglycol-diacrylate (PEG-DA) as a crosslinker, yielded a hydrogel within one hour. The elasticity E of these cross-linked gels was determined by force-indentation measurements in phosphate buffered saline (PBS) with an atomic force microscope (AFM) as described by Engler et al. Surf Sci. 2004, supra, and illustrated in FIG. 7 (see Methods). The resulting curves (see FIG. 7) were fitted with a modified Hertz model as described elsewhere, see Domke et al., 1998, supra, assuming a Poisson's ratio of v=0.45. Increasing the cross-linker/thiol ratio while keeping the HA-S concentration constant led to an increase in the Young's modulus E up to an optimal ratio above which the elasticity decreases with further increase in cross-linker concentration (FIG. 9A).

The first regime yielded a linear fit (R²=0.999) as expected from ideal rubber theory, e.g., see Flory, Principles of Polymer Chemistry. 1953, Ithaca: Cornell University Press. The decrease in elasticity beyond the optimal ratio can be explained by the bifunctional nature of PEG-DA. This crosslinker only formed an effective crosslink if both acrylate groups bound to a thiol group. Above a PEG-DA to thiol group ratio of 0.4, there were no remaining accessible thiol groups leading to dangling PEG chains that did not form effective cross-links resulting in a deviation from the linear regime and eventually to a decreased stiffness.

The second parameter that was used to tune the elasticity is the concentration of HA-S. FIG. 9B shows the elasticity of hydrogels made at varying concentrations of HA-S while maintaining a constant PEG-DA/HA-S ratio. An increase in weight percent of HA-S leads to a higher value of the Young's modulus E. The best fit for a scaling law is shown as:

E∝(c−c₀)^{α} Equation 9

with an exponent α=2.6 and a critical concentration of c₀=0.4. This is well within the range of reported exponents for other crosslinked polymer networks (Storm et al., 2005, supra).

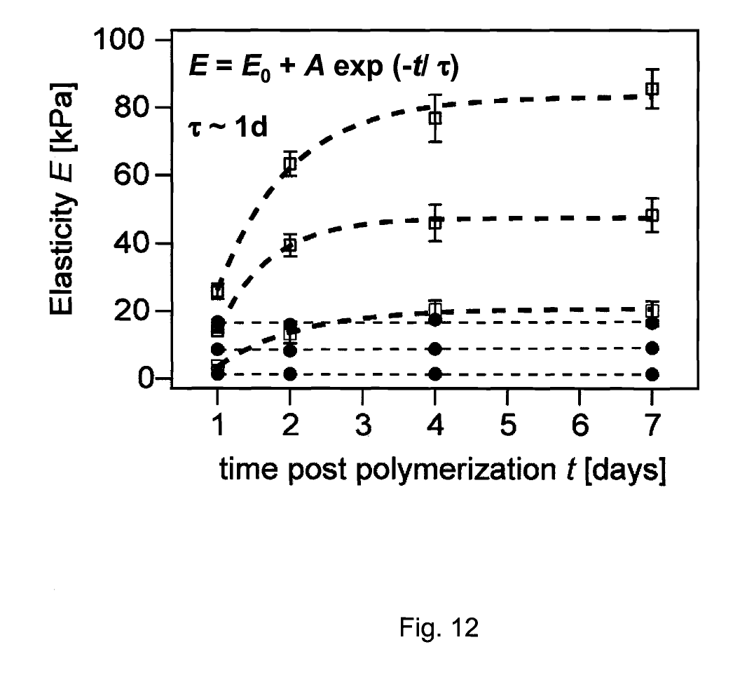

To ensure the stability of these hydrogels, the elasticity was measured several days after preparation, and initially found a significant increase of the stiffness with time as shown by the open squares in FIG. 12. This stiffening of the polymer network occurred when remaining free thiol groups formed disulfide bridges, introducing additional crosslinks. The characteristic time constant for this hardening process is about 1 day, and is reversible with DTT treatment to dissociate the disulfide bridges (data not shown). To avoid this continuous stiffening, further auto-crosslinking was prevented by treating the gels with a 1.5% solution of iodacetamide in PBS (or 2% cysteine in PBS) for two hours to cap the thiol groups. After this inactivation process the mechanical properties remained stable as depicted by the closed circles in FIG. 12. This demonstrated the tunable mechanical properties of the disclosed hydrogels in a wide range (0.1-150 kPa) for in vivo, in vitro or ex vivo use by inactivating the free thiol groups.

Accordingly, there are three parameters available to tune the Young's modulus E of the HA-S hydrogels system:the ratio of cross-linkers to thiol groups (an increase in concentration yields a linear increase in stiffness below a critical ratio), the concentration of HAS (an increase in concentration leads to an exponential increase in stiffness), and the time at which the remaining free thiol groups are inactivated (a later time leads to a stiffer gel).

### Example 4

### MSC Morphology is Dictated by Matrix Elasticity in 2D

Having characterized the mechanical properties of the HA hydrogel system, its applicability as a culture substrate for hMSCs was tested using the PA hydrogel system as a well-characterized control. Plating cells on pure HA-S gels did not result in any significant adhesion of healthy cells (data not shown), which was due to the lack of can be attributed to the repelling function HA plays in the glycocalix of cells. Pure PA gels also did not favor cell adhesion, so both substrates needed to be coated with a ligand that facilitates cell adhesion. Collagen type I can be used to coat the hydrogels for stable cell adhesion as demonstrated previously by Engler. Since the density of collagen, not only determined the number of potential receptor ligands, but also could influence the accessibility of HA, a systematic study was done of cell adhesion while varying the collagen concentration over five orders of magnitude on both PA and HA gels with a Young's modulus of E=11 kPa (see FIG. 9C). With increasing collagen concentration, the spread cell area A rises, reaching a saturation value of A=5500±482 μm² that could be fitted with a hyperbolic function:

$$A = {A}_{0} + \frac{B \cdot c}{k + c} \qquad Equation 10$$

The curves for PA and HA substrates were nearly identical, as were their respective affinity constants k_{HA}=11±3 ng cm⁻² and k_{PA}=14±6 ng cm⁻², showing that the chemical nature of the underlying elastic surface does not impact cell behavior. This is in line with an HA-binding assay we performed to compare HA and HA-S, which showed no significant recognition of the modified HA-S. As a result, collagen was used at a concentration of 20 μg cm⁻², which is well within the saturation regime.

To further elucidate potential differences between the HA gels and the standard PA system, cells were plated on substrates of different elastic modulus E, and fixed after 4 h or 24 h, and stained for actin, non-muscle myosin Ia (NMM IIa), and the nucleus. Fluorescence images were used to determine the spread cell area A and spindle factor r (or aspect ratio, which is the quotient of the major to the minor axis) of isolated cells, as described in the Methods section. The spread cell area A increased monotonically with matrix elasticity for both substrates as depicted in the left graph of FIG. 9D, approaching a maximum area Aₘₐₓ on collagen coated glass slips that were considered infinitely rigid, as compared to the cell. A simple model that assumed cell spreading depends on the polymerization of actin for growth and retrograde flow due to myosin for contraction, using the classic Hill equation provided a function for the cell area A depending on the matrix elasticity E:

$$A = {A}_{max} - \frac{B}{{k}^{m} + {E}^{m}} \qquad Equation 11$$

Fitting the data produced similar affinity constants for both gel types: k_{PA}=9.2±2 kPa and k_{HA}=8.6±1 kPa. The increase in cell area A agrees well with the prior art, and the similar behavior on the two different systems indicates that the response is purely mechanical and material independent. The other morphological trait that was analyzed was the spindle factor or aspect ratio r of the cell, which is defined as the quotient of major to minor axis of an ellipse fitted to the cell's outline. On the right side of FIG. 9D the spindle factor r is plotted versus the elasticity E of the substrates and shows a maximum around 10 kPa. Again the trend on HA gels is analogous to the PA substrates, showing very isotropic cells on soft and rigid substrates, and more elongated cells on matrices exhibiting an intermediate stiffness that is of the same order of magnitude as the cell. Fitting the aspect ratio r with a chemo-mechanical model for lineage specification proposed by Engler et al. yields similar values to the ones reported for myogenic differentiation.

All of the results above showed a similar cell behavior on HA and PA gels coated with the same collagen I concentration. This clearly indicates that the dependence on matrix elasticity E observed as increase in cell area A and a maximum of the spindle factor r on intermediate stiffness substrates (E=11 kPa) is purely a mechanical effect and is not material dependent. But is also confirmed the ability to produce a tunable, biocompatible hydrogel for controlling MSC differentiation.

### Example 5-3D Environments Using HA Hydrogels

In contrast to PA gel systems that are polymerized from toxic acrylamide monomers, the HA hydrogel system is biocompatible, allowing for encapsulation of cells during polymerization, which results in a true three dimensional environment while the mechanical properties are still well-controlled. Since cells are readily mixed with the forming hydrogel a coating procedure with the ligand, as described above, is not applicable. Rather, it is necessary to have the ligand already within the mixture and have it covalently linked to the matrix. For this purpose, 100 kDa recombinant gelatin (Fibrogen, San Francisco, Calif.) was thiol modified the same way as described for HA. Gelatin, as the heat denatured form of collagen, can still provide an attachment site for cells without forming fibrillar structures like collagen. With the added thiol groups it is covalently incorporated into the hydrogel matrix to offer ligands for the cell's integrins.

A 2D substrate was prepared with increasing amounts of gelatin to determine the optimal ligand concentration, and it was determined that there was an increase in spread cell area with higher amounts of gelatin, similar to the effect discussed regarding collagen surface density above. Furthermore, the cell morphology on 2D gels of HA coated with collagen was compared with HA mixed with gelatin (HG) at different stiffnesses (1, 11, and 34 kPa), representative of other possible concentrations. The data for cell spread area and spindle factor showed a similar behavior. The cells were also encapsulated in HG and HA gels and their proliferation was measured with a WST-1 metabolism assay, showing cells are viable and do proliferate (data not shown). Long term experiments showed viability of 3D encapsulated cells for at least up to four weeks, and when the matrix was degraded overnight with hyaluronidase the released cells started to spread normally on the glass below (data not shown). These experiments are proof of concept that the disclosed HA hydrogel is suitable as a 3D environment without sacrificing control over adhesion or elasticity. But the encapsulated cells stayed mostly round in shape since they can not readily remodel the continuous hydrogel and spread out on the short time scale. As a result, that led to a novel method that uses HG gels as a second sandwich layer on top of already spread cells to establish a conformal overlay—yielding a 3D environment.

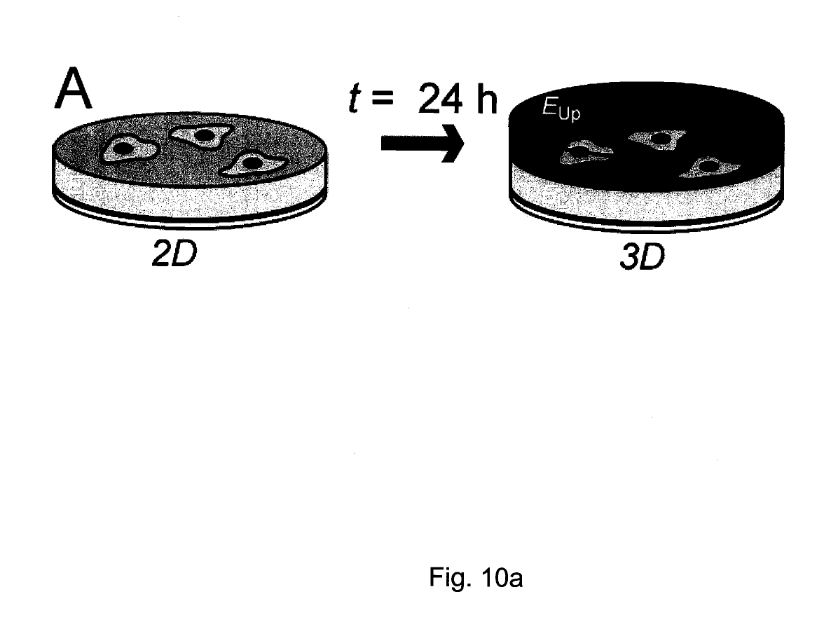

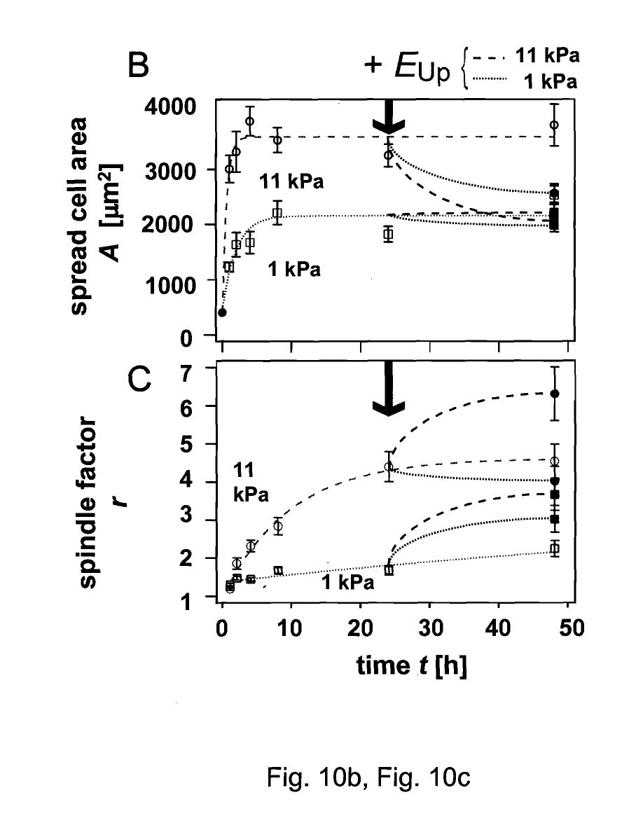

The 3D gel procedure is sketched in FIG. 10A. The cells were plated on a base hydrogel of elasticity E₀ and after time t (1 hour or 24 hours, again only representative, not limiting times), and then the second hydrogel was added of stiffness E₁ as described in the Material section.

To investigate the impact of the 3D conformal overlay on cells MCSs were cultured MSCs on 1 kPa and 11 kPa HA hydrogels for 24 hours, and a second layer of HG was added with a Young's modulus E₁ of either 1 kPa or 11 kPa. Control samples were used that were identically treated, but not coated with a hydrogel, and these control cells were analyzed in terms of cell area A and spindle factor r. FIG. 10B shows the impact of the two different sandwich gels (1 and 11 kPa) on cells plated on different base matrices (1 and 11 kPa). As shown in the graph for the aspect ratio (FIG. 10B top), adding an 11 kPa matrix on top of the cells on both matrices significantly increased the spindle factor while adding a 1 kPa matrix did not affect it significantly. Also, there was no significant difference in the two samples 1 and 11 kPa or the reverse, 11 and 1 kPa, suggesting the cell does not differentiate between top and bottom environment.

Looking at the cell area (FIG. 10C) showed a decrease for cells plated on stiff substrates (E₀=11 kPa), and no significant difference for cells on soft gels (E₀=1 kPa). To see if the increase in spindle factor was actively driven by NMM II and the actin cytoskeleton, cells were treated with blebbistatin (NMM II inhibitor) after the overlay procedure. It was observed that cells lacked any organized actin stress fibers, and yielded a smaller spindle factor, that was not significantly different from that of the 2D control. Inhibition of contractility seemed to especially affect the decrease of the minor axis that is observed for the untreated sandwich cells, thus leading to a smaller r. The overlay prevented the cell from random actin outgrowth as seen for the blebbistatin treated cell in 2D. When cells plated on 11 kPa substrates were overlaid with a hydrogel at early times (1 hour after plating), their overall shape was significantly constrained as compared to the cells in 2D, showing the kinetics of the major and the minor axis. But the cells still showed a mild increase in spindle factor r over time, yielding a similar time constant as the 2D control.

### Example 6

### Cytoskeletal Arrangement is Dictated by Matrix Elasticity in 2D and 3D

Although the spindle factor or aspect ratio r is a well established measure, it depicts only the overall cell shape and does not give any insight into cytoskeletal arrangement. But the actin/myosin network is the essential mechanical scaffold of the cell that is physically linked with the matrix and produces the mechano-response via its contractile forces. Fluorescence images of cells were immuno-stained for NMM Ia with a segmentation algorithm to calculate an order parameter. When hMSCs were cultured for 24 hours on HA gels of different elasticity, plotting the order parameter S versus the matrix elasticity E, showed low ordering for soft and rigid substrates, and a highly polarized and aligned cytoskeleton for the intermediate stiffness regime, corresponding to the typical stiffness of a cell. This phenomenon is in agreement with a theoretical model that is being described elsewhere by Zemel et al.

In plotting the order parameter in the sandwich samples, it was also shown that there is a strong dependence on the matrix elasticity that is amplified by the 3D overlay. Adding a 1 kPa overlay to the 1 kPa base gel significantly p<0.05) increased S, whereas adding it to a 11 kPa gel slightly decreased the order parameter, although not significantly. Adding a 11 kPa sandwich to the soft base matrix increased the ordering even more (p<0.01), as expected from the 2D results. Adding the same overlay to an 11 kPa gel further increased the ordering, although not significantly, which is due to the already high ordering on that base substrate (S is already close to its theoretical maximum of 1). Interestingly, the two mirror symmetric samples (1, 11 kPa) and (11, 1 kPa) are not significantly different (p>0.05), indicating that the cell does not distinguish between basal and apical gel.

### Example 7

### Nucleus Morphology is Slave to the Cytoskeletal Tension

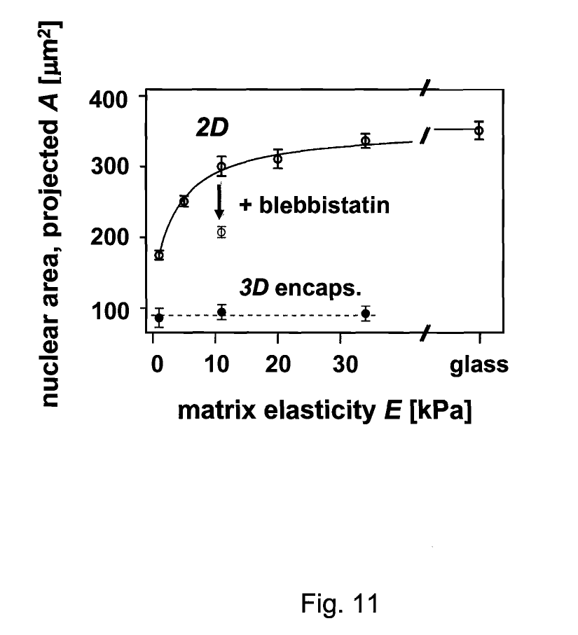

Since matrix elasticity not only influences cell and cytoskeleton morphology, but also the differentiation of hMSCs, and therefore global changes in patterns of gene expression, the morphology of the nucleus was analyzed in terms of projected area A and spindle factor of the projected area r to gain insight into the transduction of mechanical signals from the matrix to the nucleus. Both parameters showed an astonishingly similar behavior to the overall cell morphology. The projected nuclear area A rose with increasing matrix elasticity and the spindle factor was highest on substrates with a Young's modulus E=11 kPa (FIG. 11).

Cells encapsulated in 3D gels do not exhibit a elasticity dependent behavior and have a much smaller cross-sectional area as indicated with the broken line within the SEM region. Fluorescence micrographs best represented the average values. The increase in projected area can be explained by the increasing cytoskeletal tension that is compressing the nucleus, while the change in aspect ratio is due to the anisotropic polarization of the cytoskeleton imposing the same anisotropy onto the nucleus. This phenomenon can be by inhibited by blebbistatin treatment that decreases the tension of the cell as shown exemplary for 11 kPa substrates.

While others have studied nuclear deformation, this is the first demonstration of the systematic change in morphology that is purely due to variation of cytoskeletal tension by the substrate elasticity. This connection between cytoskeletal tension and nuclear mechanical deformation suggests a mechanism whereby substrate stiffness can directly impact transcription and therefore differentiation as speculated in a review by Dahl et al., Circulation Research, 102(11):1307-1318 (2008). See also Pajerowski et al., Proc. Natl. Acad. Sci. USA 104(40):15619-15624 (2007) showing nuclei in human embryonic stem cells are highly deformable and stiffen 6-fold during terminal differentiation. Although the differences are not as pronounced as the observation for the cell shape, the nuclear morphology also varies with matrix elasticity, with the aspect ratio r being between 1.4 and 1.75, peaking at a matrix elasticity of 11 kPa, following the same trend.

Mechano-biology is a broad field encompassing the recognition in the present invention that most tissue cells not only adhere to, but also pull on, their microenvironment, and as a result anchorage-dependent cells respond to the stiffness of the underlying matrix in ways that relate to tissue elasticity. In some aspects, microenvironments that are too soft or too stiff have implications in disease, as well as development, and highlight the need to understand the important role provided herein, for matrix physical properties in 2D and 3D, and how cells feel the cellular matrix. The present findings demonstrate how to finely tune the mechanical parameters of hydrogels formed with thiol-modified hyaluronic acid to cover the whole range that is physiologically relevant. Comparison of cell morphology on PA and HA substrates showed similar behavior indicating that the mechano-sensitivity of cells is a general physical feature that remains material independent. The thiol functionalities allow for coupling of a variety of molecules to the matrix as demonstrated here with thiol-modified gelatin and the maleimide dye.

Analysis of the cell morphology in 3D shows that elasticity is the primary factor dictating the shape and is amplified by adding the three dimensional overlay. When cells are conformally overlaid at an early timepoint the additional sandwich layer constrains the shape since the cell is not able to remodel its environment on short timescales (1 day). Analysis of the cytoskeletal arrangement also shows a strong dependence on matrix elasticity and cells do not exhibit significant differences whether the stiffer matrix is the lower or upper gel if sandwiched between a soft and a stiff gel. When cell shape is constrained by the overlay, the cells still develop a highly polarized cytoskeleton, although there shape stays isotropic, showing that it is essential to control the mechanical properties of the micro-environment of cells. Using different elasticities for the two gel layers can be used as an in vitro model for the interface of diseased and healthy tissue, where different elasticities coexist. Further addition of different ligands in the two layers permits differential cell behavior towards different biochemical compositions while maintaining a well-defined elasticity, permitting an in vitro reconstruction of micro-environments, i.e., basement membranes. Consequently, in addition to the regulation of differentiation of anchorage-dependent mesenchymal stem cells, many applications will result from the recognition that tissue cells feel and respond to the mechanics (elasticity) of their substrate, e.g., modified strategies for tissue repair and cell scaffolding, such as the development of fibrous scaffolds for cell seeding.

All patents, patent applications and publications referred to in the present specification are also fully incorporated by reference.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the basic principles of the invention. Such modifications and additional embodiments are also intended to fall within the scope of the appended claims.

What is claimed is:

1. A method for regulating differentiation of anchorage-dependent cell, comprising:
providing a biocompatible hydrogel substrate having an elasticity defined by elastic constant E;
introducing the anchorage-dependent cell onto said substrate; and
developing the anchorage-dependent cell into a differentiated cell type, wherein lineage commitment is effected by the elasticity of the underlying substrate.

2. The method of claim 1, wherein the substrate comprises an ultra-thin layer gel matrix and the method further comprises setting the elasticity of the substrate by selecting a concentration of cross-linking density within the matrix, such that E is adjustable over several orders of magnitude, from extremely soft to stiff.

3. The method of claim 2, wherein the hydrogel is prepared using high molecular weight hyaluronic acid (HA), polymerized without toxicity to the cells.

4. The method of claim 3, wherein the substrate comprises a tunable matrix formed by the step of employing a monofunctional thiol modification of HA to achieve a finely tunable Young's elasticity modulus E of the substrate that ranges from 0.1 to 150 kPa.

5. The method of claim 4, further comprising inactivating residual thiol groups after optimal E has been achieved in the thiol-modified hydrogel, thereby stabilizing the gel.

6. The method of claim 4, wherein the anchorage-dependent cell is a pluripotent mesenchymal stem cell.

7. The method of claim 6, wherein the pluripotent mesenchymal stem cell is a human pluripotent mesenchymal stem cell.

8. The method of claim 6, wherein the anchorage-dependent cell differentiates into a neurogenic, myogenic or osteogenic-type cell.

9. The method of claim 1, wherein cell adhesions provide necessary attachments permitting the cell to feel its microenvironment, and adhesion area increases linearly with E, such that larger deformation within the cell occurs on stiffer matrices and larger deformation in the substrate occurs on softer matrices.

10. The method of claim 1, further comprising regulating cell shape and differentiation by controlling cell strain, such that there is an inverse relationship between intracellular and extracellular strains so that on stiff matrices, cell strains are large, while matrix strains are small, and on soft matrices, cell strains are small, while matrix strains are large.

11. The method of claim 10, further comprising regulating nucleus morphology of the cell as a function of matrix elasticity.

12. A cell substrate for anchorage-dependent cell culture, the substrate comprising a tunable matrix formed by the step of employing a monofunctional thiol modification of HA to achieve a finely tunable Young's elasticity modulus E of the substrate that ranges from 0.1 to 150 kPa.

13. The cell substrate of claim 12, wherein the thiol functionalities allow for coupling of one or more additional molecules to the matrix.

14. The method of using the HA hydrogel of claim 12 as a tunable, biocompatible 3D environment for controlling differentiation of cultured anchorage dependent cells, while maintaining control over elasticity and cell adhesion, and retaining cell viability for at least 4 weeks.

15. The stabilized cell substrate for anchorage-dependent cell culture of claim 14, wherein residual thiol groups are inactivated after optimal E has been achieved in the thiol-modified hydrogel.

16. The method of using the HA hydrogel of claim 15 as a tunable, biocompatible 3D environment for controlling differentiation of cultured anchorage dependent cells, while maintaining control over elasticity and cell adhesion, and retaining cell viability for at least 4 weeks.

17. A method of forming 3D HA sandwich hydrogel gel as used in claim 14, the method comprising plated anchorage dependent cells on a base hydrogel of elasticity E₀, and after time t overlaying a second homogeneous hydrogel layer of stiffness E1.

18. The method of stabilizing the 3D HA sandwich hydrogel gel of claim 17 by inactivating residual thiol groups after optimal E has been achieved in the thiol-modified 3D hydrogel.
